(19) United States
(12) Patent Application Publication
(10) Pub. No.: US 2024/0141270 A1
(43) Pub. Date: May 2, 2024

(54) METHODS, APPARATUS, AND SYSTEMS FOR USING THREE-DIMENSIONAL PRINTERS TO FORM DISCRETE BIOMASS RECEPTACLES USABLE TO PERFORM PROCESSES INCLUDING CULTIVATING AND/OR PROCESSING ALGAE FOR BIOFUEL PRODUCTION

(71) Applicant: Deep Green Biomass LLC, New York, NY (US)

(72) Inventors: Peter Kim, Kuala Lumpur (MY); Bret Crochet, San Diego, CA (US); Kevin Brooks, Destin, FL (US)

(73) Assignee: Deep Green Biomass LLC, New York, NY (US)

(21) Appl. No.: 18/328,767

(22) Filed: Jan. 18, 2024

Related U.S. Application Data
(63) Continuation of application No. 17/673,783, filed on Feb. 17, 2022.

(51) Int. Cl. C12M 1/00; C10L 1/02; C12M 1/34; C12M 1/36; C12M 3/00; C12N 1/12; C12P 7/06

(52) U.S. Cl. CPC C12M 21/12; C10L 1/02; C12M 21/02; C12M 23/40; C12M 23/44; C12M 41/36; C12M 41/48; C12N 1/12; C12P 7/065; C10L 2290/26

(57) ABSTRACT

Some embodiments are directed to a method of manufacturing discrete biomass receptacles with at least one of smooth and faceted sides that are usable to perform processes including cultivating algae for biofuel production and/or processing algae for biofuel production. The method can include: creating an algae based material from cultivating algae for biofuel production and/or processing algae for biofuel production; heating the algae based material; and extruding the algae base material using a three-dimensional printer, the extruding including extruding the algae based material in a concentric pattern in the z-axis to form the biomass receptacles, such that the heating of the algae based material enhances the extrusion.

### BACKGROUND

Some embodiments are directed to methods, apparatus, and systems for efficient harvesting and cultivation of microalgae biomass techniques, such as in the context of generating biofuel, and in particular biofuel sourced from algae, for example. Some of these embodiments are directed to using three-dimensional printers to form discrete biomass receptacles usable to perform processes including cultivating and/or processing algae for biofuel production.

There is considerable interest in the development of renewable energy sources to replace petroleum-based fuels. It has been discovered that certain algae have a large oil or lipid content, and thus provide a source for the production of biodiesel. However, there is a lack of efficient and cost-effective algal biomass production systems. Open pond technology is often expensive and susceptible to contamination. Current closed photobioreactors using fiber optic light transmission can be prohibitively expensive.

### SUMMARY

Therefore, it may be beneficial to provide devices and methods for generating biofuel and other forms of energy from algae. It may be especially beneficial for such techniques to provide sufficient illumination to algae cultures to support growth. Further, it may be beneficial for these approaches to provide the required nutrients and gases to support algal growth in an efficient manner. These techniques may also provide for the removal of oil from algae cultures.

Some embodiments provide an improved approach to the cultivation and harvesting of biomass to create biofuels. An exemplary cultivation system includes discrete biomass receptacles, mixotrophic growth, a system of pipes providing an individual pipe to each receptacle, a sensor module within each pipe able to detect a plurality of conditions, and a system for relaying information to a controller.

Discrete biomass receptacles are a structure having an exterior surface and an interior surface, the interior surface defining an isolated space configured to retain biomass and cultivation media, are freestanding, and without an enclosed upper surface. The receptacles are clustered together. The receptacles do not exchange biomass or cultivation media with other receptacles prevent the spread of contaminants beyond individual receptacles.

Mixotrophic growth occurs when photoautotrophic growth and heterotrophic growth are performed simultaneously by biomass within each of the receptacles. Mixotrophic growth provides many benefits over solely photoautotrophic growth, including increased yield, photoinhibition protection, and prolonged exponential growth phases.

A system of pipes providing an individual pipe to each receptacle enables individual treatment for each receptacle. The pipes are able to dispense growth media that affects the biomass cultivation media in order to increase yield. Sensors within each pipe for each receptacle relay information regarding a number of conditions in the biomass cultivation media.

A controller is able to affect change to as many or as little receptacles as needed to increase growth. A controller can operate autonomously or be controlled manually.

In another aspect, the present disclosure is directed to a system for harvesting biomass utilizing a mobile overhead harvesting machinery, multiple dewatering processes, and multiple oil extraction processes.

Mobile overhead harvesting machinery harvests by lowering harvesting baskets into individual receptacles containing biomass. Mobile overhead harvesting machinery harvests from one section of a receptacle cluster and then moves on to the next. An integrated nutrient delivery system also dispenses nutrients and replenishes water when the harvesting basket enters the receptacle. The harvesting basket also contains sensors which detect a plurality of biomass conditions. The overhead harvesting machinery can selectively harvest and can treat each receptacle differently. The overhead harvesting machinery is affected by a controller with automated actions or manual controls.

Once the biomass is harvested and moved to the extraction area by conveyors, a mechanical belt press dewaters the algae, removing 80% of the water. The removed water is recycled back into the cultivation system. The dewatered algal biomass is then moved to an ultrasonic reactor in which solvent extraction occurs using methanol and chloroform. The mixture of algal biomass, oil, and solvent is moved to a filter press which separates dry and wet material. Dry algae cake and an oil and solvent mixture are produced. The oil and solvent mixture then undergoes distillation, separating the oil and solvent, producing crude algae oil. The solvent is cooled and recycled back into the ultrasonic reactor to be used again.

Thus, some embodiments are directed to a method of manufacturing discrete biomass receptacles with at least one of smooth and faceted sides that are usable to perform processes including cultivating algae for biofuel production and/or processing algae for biofuel production. The method can include: creating an algae based material from cultivating algae for biofuel production and/or processing algae for biofuel production; heating the algae based material; and extruding the algae base material using a three-dimensional printer, the extruding including extruding the algae based material in a concentric pattern in the z-axis to form the biomass receptacles, such that the heating of the algae based material enhances the extrusion.

In some of these embodiments, the extruding step includes forming a bottom of the biomass receptacles separately from a remainder of the biomass receptacles.

Some of these embodiments further include adding the bottom to the remainder of each biomass receptacles to form complete biomass receptacles usable for cultivating algae for biofuel production and/or processing algae for biofuel production.

In some of these embodiments, the extruding includes forming at least one of an exterior biomass receptacle component and an interior lining for the biomass receptacle.

In some of these embodiments, the extruding includes forming at least one of an exterior biomass receptacle component and an interior lining for the biomass receptacle from non-algae material.

Some of these embodiments further include, during production or postproduction, forming the exterior receptacle component to be tinted red or blue in order to promote photoautotrophic algae growth within the biomass receptacles.

Some of these embodiments further include embedding within the exterior biomass receptacle component at least one of tubes, sensors and associated wires, harnesses, and guide paths.

Some of these embodiments further include embedding within the internal receptacle liner at least one of tubes, sensors and associated wires, harnesses, and guide paths.

### BRIEF DESCRIPTION OF THE DRAWINGS

Various exemplary aspects of the systems and methods will be described in detail, with reference to the following figures, wherein:

FIG. 1 is a schematic of an exemplary process for mixotrophic growth and the interactions between photoautotrophic growth and heterotrophic growth, in accordance with some embodiments of the present disclosure;

FIG. 2A-B are schematics of a section of an exemplary system for cultivating algae biomass using tanks with a shape such that each tank is a discreet cultivation environment, in accordance with some embodiments of the present disclosure;

FIG. 3 is a schematic of a piping assembly used in biomass cultivation, in accordance with some embodiments of the present disclosure;

FIG. 4 is a schematic of an exemplary process for maintenance and optimization of biomass cultivation, in accordance with some embodiments of the present disclosure;

FIG. 5 is a schematic of a lining that could be installed on the inner surface of biomass receptacle is illustrated, in accordance with some embodiments of the present disclosure;

FIG. 6A-B are schematics of an exemplary method for harvesting biomass from a number of biomass receptacles using a mobile overhead harvesting machine and harvesting baskets, in accordance with some embodiments of the present disclosure;

FIG. 7 is a schematic of an exemplary process for the cultivation, harvesting, and extraction of crude algae oil, in accordance with some embodiments of the present disclosure; and

FIG. 8 is a schematic of an exemplary process for maintenance and optimization of harvesting biomass from a number of biomass receptacles using a mobile overhead harvesting machine, in accordance with some embodiments of the present disclosure.

### DETAILED DESCRIPTION

These and other features and advantages are described in, or are apparent from, the following detailed description of various exemplary embodiments.

The Detailed Description is provided via the following headings:
1. CULTIVATION OF BIOMASS
   1A. MIXOTROPHIC ALGAE
   1B. A SCALABLE SYSTEM FOR ALGAE POOLS
   1C. LIGHTING OPTIMIZATION
   1D. ADJUSTMENT OF ALGAE METABOLISM
   1E. NUTRIENTS AND WATER DELIVERY SYSTEM
2. HARVESTING OF BIOMASS
   2A. FLOCCULATION OF BIOMASS
   2B. MOBILE OVERHEAD HARVESTING MACHINERY AND HARVESTING BASKET
   2C. OVERVIEW OF ENTIRE HAPP PROCESS

It will be understood that when an element is referred to as being “on”, “connected”, or “coupled” to another element, it can be directly on, connected, or coupled to the other element or intervening elements that may be present. In contrast, when an element is referred to as being “directly on”, “directly connected”, or “directly coupled” to another element, there are no intervening elements present. As used herein the term “and/or” includes any and all combinations of one or more of the associated listing items. Further, it will be understood that when a layer is referred to as being “under” another layer, it can be directly under or one or more intervening layers may also be present. In addition, it will also be understood that when a layer is referred to as being “between” two layers, it can be the only layer between the two layers, or one or more intervening layers may also be present.

It will be understood that, although the terms “first”, “second”, etc. may be used herein to describe various elements, components, regions, layers, and/or sections, these elements, components, regions, layers and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of exemplary embodiments.

In the drawing figures, the dimensions of layers and regions may be exaggerated for clarity of illustration. Like reference numerals refer to like elements throughout. The same reference numbers indicate the same components throughout the specification.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For exemplary, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

The terminology used herein is for the purpose of describing particular embodiments only and is not intended to be limiting of exemplary embodiments. As used herein, the singular forms “a”, “an”, and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

Exemplary embodiments are described herein with reference to cross-sectional illustrations that are schematic illustrations of idealized embodiments (and intermediate structures) of exemplary embodiments. As such, variations from the shapes of the illustrations as a result, for exemplary, of manufacturing techniques and/or tolerances, are to be expected. Thus, exemplary embodiments should not be construed as limited to the particular shapes of regions illustrated herein but are to include deviations in shapes that result, for exemplary, from manufacturing. For exemplary, an implanted region illustrated as a rectangle will, typically, have rounded or curved features and/or a gradient of implant concentration at its edges rather than a binary change from implanted to non-implanted region. Likewise, a buried region formed by the implantation may result in some implantation in the region between the buried region and the surface through which the implantation takes place. Thus, the regions illustrated in the figures are schematic in nature and their shapes are not intended to illustrate the actual shape of a region of a device and are not intended to limit to scope of exemplary embodiments.

Unless otherwise defined, all terms (including technical and scientific terms) used herein have the same meaning as commonly understood by one of ordinary skill in the art to which exemplary embodiments belong. It will be further understood that all terms, such as those defined in commonly-used dictionaries, should be interpreted as having a meaning that is consistent with their meaning in the context of the relevant art and will not be interpreted in an idealized or overly formal sense unless expressly so defined herein. As used herein, expressions such as “at least one of”, when preceding a list of elements, modify the entire list of elements and do not modify the individual elements of the list.

When the terms “about” or “substantially” are used in this specification in connection with numerical values, it is intended that the associated numerical value include a tolerance of ±10% around the stated numerical value. Moreover, when reference is made to percentages in this specification, it is intended that those percentages are based on weight, i.e., weight percentages. The expression “up to” includes amounts of zero to the expressed upper limit and all values therebetween. When ranges are specified, the range includes all values therebetween such as increments of 0.1%. Moreover, when the words “generally” and “substantially” are used in connection with geometric shapes, it is intended that the precision of the geometric shape is not required but that latitude for the shape is within the scope of the disclosure. Although the tubular elements of the embodiments may be cylindrical, other tubular cross-sectional forms are contemplated, such as square, rectangular, oval, triangular, and others.

Although corresponding plan views and/or perspective views of some cross-sectional view(s) may not be shown, the cross-sectional view(s) of device structures illustrated herein provide support for a plurality of device structures that extend along two different directions as would be illustrated in a plan view, and/or in three different directions as would be illustrated in a perspective view. The two different directions may or may not be orthogonal to each other. The three different directions may include a third direction that may be orthogonal to the two different directions. The plurality of device structures may be integrated in a same electronic device. For exemplary, when a device structure (e.g., a memory cell structure or transistor structure) is illustrated in a cross-sectional view, an electronic device may include a plurality of the device structures (e.g., memory cell structures or transistor structures), as would be illustrated by a plan view of the electronic device. The plurality of device structures may be arranged in an array and/or in a two-dimensional pattern.

Reference will now be made in detail to embodiments, examples of which are illustrated in the accompanying drawings, wherein like reference numerals refer to the like elements throughout. In this regard, the present embodiments may have different forms and should not be construed as being limited to the descriptions set forth herein. Accordingly, the embodiments are merely described below, by referring to the figures, to explain exemplary embodiments of the present description.

### 1. Cultivation of Biomass

### 1a. Mixotrophic Algae

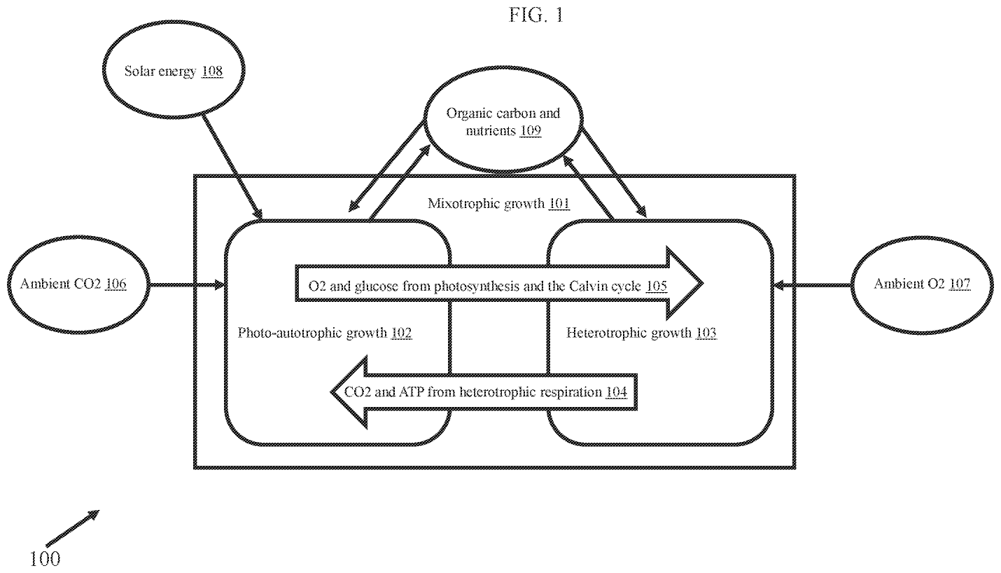

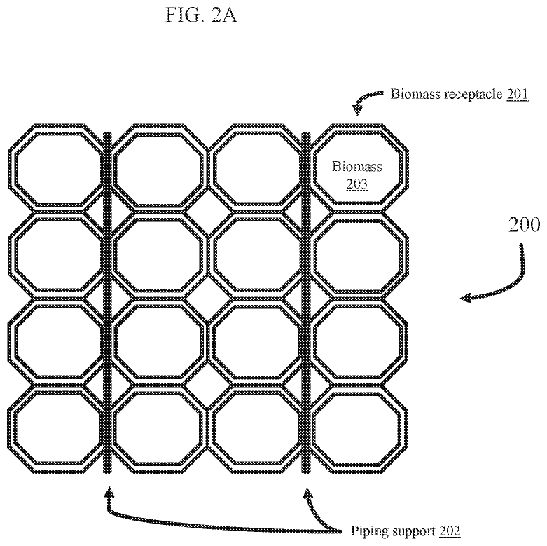

Referring to FIG. 1, a block diagram of an exemplary mixotrophic system 100 is illustrated, in accordance with some embodiments of the present disclosure. In mixotrophic growth 101 the two metabolic processes, photosynthesis for photoautotrophic growth 102 and aerobic respiration for heterotrophic growth 103, affect each other, contributing to synergistic effects and improving the growth rate, with an enhancement of biomass productivity.

Certain aspects of cellular metabolism are discussed in “Oxygen Is the High-Energy Molecule Powering Complex Multicellular Life: Fundamental Corrections to Traditional Bioenergetics” by Schmidt-Rohr, K. (2020), the entire contents of which are incorporated herein by reference for all purposes. Photo-autotrophic growth 102 occurs when microalgae utilize ambient carbon dioxide (CO2) 106, CO2 and adenosine triphosphate (ATP) produced by heterotrophic respiration 104, solar energy 108, and organic carbon and nutrients 109 to perform photosynthesis and generate oxygen (O2) and glucose 105, and regenerate organic carbon and nutrients 109. Biomass cultivation using solely photoautotrophic species requires a large supply of ambient carbon dioxide (CO2) 106 and creates a significant bottleneck for large scale algal cultivation. Mixotrophic growth 101 mitigates this problem by providing an endogenic source of CO2 and ATP generated by heterotrophic growth.

Heterotrophic growth 103 occurs when microalgae utilize ambient O2 107, O2 and glucose from photosynthesis 105, and organic carbon and nutrients 109 to perform heterotrophic respiration and generate CO2 and ATP 104, and regenerate organic carbon and nutrients 109.

O2 and glucose from photosynthesis and the Calvin cycle 105 are the outputs of photo-autotrophic growth 102 and are inputs of heterotrophic growth 103. Photosynthesis is the first step in the process of photoautotrophic growth 102 and begins when one molecule of the pigment chlorophyll absorbs one photon and loses one electron. This electron is passed to a modified form of chlorophyll called pheophytin, which passes the electron to a quinone molecule, starting the flow of electrons down an electron transport chain that leads to the ultimate reduction of NADP to NADPH. In addition, this creates a proton gradient (energy gradient) across the chloroplast membrane, which is used by ATP synthase in the synthesis of ATP. The chlorophyll molecule ultimately regains the electron it lost when a water molecule is split in a process called photolysis, which releases a O2 molecule as a waste product. The overall equation is: 2 H2O+2 NADP++3 ADP+3 Pi+light→2 NADPH+2 H++3 ATP+O2.

The next step of photoautotrophic growth 102 is the Calvin cycle. In the Calvin cycle, the enzyme RuBisCO captures CO2 from the atmosphere and uses the NADPH and ATP from photosynthesis and releases three-carbon sugars, which are later combined to form glucose. The overall equation for the light-independent reactions in green plants is: 3 CO2+9 ATP+6 NADPH+6 H+→C3H6O3-phosphate+9 ADP+8 Pi+6 NADP++3 H2O.

Carbon fixation produces the intermediate three-carbon sugar product, which is then converted into the final carbohydrate products. The fixation or reduction of carbon dioxide is a process in which carbon dioxide combines with a five-carbon sugar, ribulose 1,5-bisphosphate, to yield two molecules of a three-carbon compound, glycerate 3-phosphate, also known as 3-phosphoglycerate. Glycerate 3-phosphate, in the presence of ATP and NADPH produced during the light-dependent stages, is reduced to glyceraldehyde 3-phosphate. This product is also referred to as 3-phosphoglyceraldehyde (PGAL) or, more generically, as triose phosphate. Most (5 out of 6 molecules) of the glyceraldehyde 3-phosphate produced is used to regenerate ribulose 1,5-bisphosphate so the process can continue. The triose phosphates not thus “recycled” often condense to form hexose phosphates, which ultimately yields glucose.

CO2 and ATP from heterotrophic respiration 104 are the outputs of heterotrophic growth 103 and are inputs of photo-autotrophic growth. The heterotrophic respiration performed as described here is aerobic. Aerobic respiration requires oxygen (O2) in order to create ATP. Although carbohydrates, fats, and proteins are consumed as reactants, aerobic respiration is the preferred method of pyruvate breakdown in glycolysis, and requires pyruvate to the mitochondria in order to be fully oxidized by the citric acid cycle. The products of this process are carbon dioxide and water, and the energy transferred is used to break bonds in ADP to add a third phosphate group to form ATP (adenosine triphosphate), by substrate-level phosphorylation, NADH and FADH2. The simplified reaction is as follows: C6H12O6+6 O2→6 CO2+6H2O+heat.

The potential of NADH and FADH2 is converted to more ATP through an electron transport chain with oxygen and protons (hydrogen) as the “terminal electron acceptors”. Most of the ATP produced by aerobic cellular respiration is made by oxidative phosphorylation. The energy of O2 released is used to create a chemiosmotic potential by pumping protons across a membrane. This potential is then used to drive ATP synthase and produce ATP from ADP and a phosphate group. Current estimates for ATP yield range around 29 to 30 ATP per glucose. Aerobic metabolism is up to 15 times more efficient than anaerobic metabolism (which yields 2 molecules ATP per 1 molecule glucose) because the double bond in O2 is of higher energy than other double bonds or pairs of single bonds in other common molecules in the biosphere.

Certain aspects of mixotrophic growth are discussed within Prospects and Challenges in Algal Biotechnology edited by Bhumi Nath Tripathi, Dhananjay Kumar, Ch 5 pp. 141-177, Springer, Nov. 8, 2017, the entire contents of which are incorporated herein by reference for all purposes. Mixotrophic growth 101 allows microalgae to live via photo-autotrophy and heterotrophy using both inorganic and organic carbon sources. The assimilation of organic carbon is influenced by light and occurs through photosynthesis, while the assimilation of organic compounds takes place through aerobic respiration, which depends on the availability of organic carbon. Microalgae cultivated under mixotrophic mode showed an improve in their growth rate, a reduction in growth cycle and biomass losses in dark hours and an augmentation in biomass productivity. Heterotrophic algae are able to continue biomass production during the night because heterotrophic growth is not dependent on light, unlike photoautotrophic algae. Having species that are still undergoing the same level of growth at night compensates for the lost biomass production that occurs at night with photoautotrophic algae.

Some strains, under mixotrophic condition, are able to enhance lipid content as percentage on dry weight obtaining a lipid productivity useful for microalgal biodiesel production. CO2 emitted by microalgae through aerobic respiration can be caught and reused by microalgae mixotrophically cultivated. This mechanism enhances inorganic carbon availability for microalgae and thus further improves biomass and lipid productivities.

In solely photo-autotrophic conditions a further increase in light level, not only does not increase photosynthesis, but also reduces the biomass growth rate. This phenomenon is called photoinhibition. Microalgae are photo-inhibited at light intensities little higher than the light level at which the specific growth rate peaks. Removing photoinhibition or its deferment to higher light intensities can significantly increase the average daily growth rate of algal biomass. Because of light saturation, and subsequent photoinhibition, the biomass growth rate, and thus total yield, is much lower than theoretical calculations. It is generally assumed that photoinhibition results from two factors. The first is the incapability of the photosynthetic apparatus to employ the exceeding light energy absorbed by the photosynthetic antenna: there is a disparity between the quick rate of photon catch by the light-harvesting apparatus and the slower downstream rate of photosynthetic electron transfer. The second is the production of reactive oxygen species (ROS). Due to its electron configuration, O2 has a very high oxidizing potential and is the final electron acceptor in aerobic respiratory electron transport. Oxygenic photosynthesis uses photon energy to produce sugar from CO2 and H2O, and releases O2 as a waste product. The production and consumption of energy by photosynthetic electron transport and the Calvin-Benson cycle becomes unbalanced without sufficient CO2. Excess photon energy causes the production of reactive oxygen species (ROS), which trigger oxidative damage to Photosystem II and Photosystem I.

Certain aspects of photoinhibition resistance are discussed in Doubling of Microalgae Productivity by Oxygen Balanced Mixotrophy. ACS Sustainable Chem. Eng. 2020, 8, 15, 6065-6074, the entire contents of which are incorporated herein by reference for all purposes. Microalgae cultivated in mixotrophic conditions are less sensitive to photoinhibition than those cultivated in photoautotrophic mode. Moreover, when photoinhibition does manage to occur, microalgae under mixotrophic cultivation recover more quickly and to a higher extent. The reduced sensibility of mixotrophic cells to photoinhibition is caused by multiple mechanisms.

One reason mixotrophic conditions lead to photoinhibition-resistant microalgae is re-balance of light-dependent and enzymatic-dependent reaction. The light-catch reaction is quicker than the subsequent enzyme-mediated reactions, thus the maximum rate of photosynthesis must be checked by the concentration of one of the Calvin cycle's enzymes. A deficiency of electron sinks downstream of photosystem I (e.g. carbon fixation) can result in accumulation of electrons in the electron transport flow and consequently an increased risk of ROS production and photoinhibition. An increase in Calvin cycle activity, due to the abundance of organic carbon, can lead to increase in the consumption of reduction power.

Another reason mixotrophic conditions lead to photoinhibition-resistant microalgae is rapid repair of damage to photosystem II. Damages from photoinhibition are promptly repaired, depending on the environmental conditions and the physiological conditions of the cell, through the action of D1 protein. Photosystem II is susceptible to be damaged by high irradiation. It has been suggested that the turnover of D1 protein is part of a repair system to replace the damaged function centers with newly synthesized protein D1 thus restore the normal Photosystem II activity. The reestablishment from the photoinhibition is not just a counter reaction to stressing condition, but it is an active anabolic process aimed to synthesize great amount of D1 protein. The more rapid recovery rate founded in mixotrophic cultures is ascribed to higher metabolic activity.

Yet another reason mixotrophic conditions lead to photoinhibition-resistant microalgae is reduction in the dimension of the light-harvesting antenna and reduction in chlorophyll content. This mechanism reduces the light adsorbing capacity of individual cells, increasing light penetration in deep layers of biomass and reducing heat dissipation of absorbed light energy, thereby increasing photosynthetic efficiency in high light and high cell density culture. This mechanism is highly effective considering not the individual efficiency, but the whole production system (high densities PBR). The less effective performance of an individual cell protects the cell and distributes light thus achieving a better performance of the whole system.

Lastly, one more reason mixotrophic conditions lead to photoinhibition-resistant microalgae is increased oxygen consumption. The oxygen produced by photosynthesis is released in the culture medium. Some microalgae strains are not able to survive in significantly O2-oversaturated environment longer than 2-3 h. Cells growing mixotrophically, thanks to the respiration reactions promoted by carbon abundance, consume oxygen and allow a considerable decrease of the concentration of dissolved oxygen in the cultivation environment, thus reducing photooxidative damage.

As described by Scott J. Edmundson and Michael H. Huesemann in “The dark side of algae cultivation: Characterizing night biomass loss in three photosynthetic algae, Chlorella sorokiniana, Nannochloropsis salina and Picochlorum sp.” (2015), mixotrophic growth 101 also reduces the lost biomass from respiration at night. The quantification of biomass productivity is not as straightforward as simply measuring the growth rate of an alga strain. Many environmental and physiological factors influence the ultimate yield of captured solar energy in algal biomass. Substantial respiratory losses at night, for example, can significantly impact the biomass production capacity of solar-based algae cultures. Night biomass loss in photosynthetic algae is an essential parameter that is often overlooked when modeling or optimizing biomass productivities. Night respiration acts as a tax on daily biomass gains. The dynamics of biomass loss should be taken into consideration in algae strain selection, are critical in predictive modeling of biomass production based on geographic location and can influence the net productivity of photosynthetic cultures used for bio-based fuels or products. Greater than 30% of the biomass fixed during the day in outdoor, sunlit algae cultures (both ponds and photobioreactors) can be lost at night.

Mixotrophic growth 101 also provides prolonged exponential growth phases with phase is one of five algae growth phases and is also the most important for biomass cultivation.

Certain aspects of algae growth phases are discussed in Fogg, G. E. and Thake, B. (1987) Algae Cultures and Phytoplankton Ecology. 3rd Edition, The University of Wisconsin Press Ltd., London, the entire contents of which are incorporated herein by reference for all purposes. The first phase, which does not always occur is the lag phase. The condition of the inoculum has a strong bearing on the duration of the lag phase. An inoculum taken from a healthy exponentially growing culture is unlikely to have any lag phase when transferred to fresh medium under similar growth conditions of light, temperature, and salinity. In general, the length of the lag phase will be proportional to the length of time the inoculum has been in phases three-five. A lag phase may also occur if the inoculum is transferred from one set of growth conditions to another.

The second phase is the exponential phase and is the phase most related to growth rate measurement. The growth rate of a microalgal population is a measure of the increase in biomass over time and it is determined from the exponential phase. Growth rate is one important way of expressing the relative ecological success of a species or strain in adapting to its natural environment or the experimental environment imposed upon it. The duration of exponential phase in cultures depends upon the size of the inoculum, the growth rate, and the capacity of the medium and culturing conditions to support algal growth. Biomass estimates need to be plotted over time, and logistical constraints determine their frequency but once every one to two days is generally acceptable. Cell count and dry weight are common units of biomass determination. In-vivo fluorescence and turbidity can be used as surrogate measures which enable higher temporal resolution due to the logistical ease of measurement (correlations between fluorescence or turbidity and cell count can be established but they will become less accurate as experimental conditions are varied. For example, cell fluorescence may vary with temperature so an experiment with several test temperatures may need correlations to be determined for each temperature. Correlations also become inaccurate as cultures move into stationary phase so fluorescence cannot be used as a substitute for cell counts where an estimate of final cell yield is needed). Once the growth phase has been plotted (time on x-axis and biomass on logarithmic y-axis) careful determination of the exponential phase of growth is needed.

For healthy cells of a robust species, small inoculums equal to 0.5% of the volume of the new culture will normally generate new healthy cultures. If the species is delicate or the culture less healthy then a larger inoculum of ˜10% may be needed to support a new culture.

Declining growth is the next phase of algae growth, and the methods and systems currently disclosed ensure the cultivating microalgae do not enter this phase. Declining growth normally occurs in cultures when either a specific requirement for cell division is limiting or something else is inhibiting reproduction. In this phase of growth, biomass is often very high and exhaustion of a nutrient salt, limiting carbon dioxide or light limitation become the primary causes of declining growth. When biomass is increasing exponentially a constant supply of air (or air plus CO2) will only be in balance with growth at one point during exponential phase. At low cell densities too much CO2 may lower the pH and depress growth. CO2 limitation at high cell densities causes any further biomass increases to be linear rather than exponential (with respect to time) and proportional to the input of CO2. The use of mixotrophic growth ensures that O2 and CO2 levels are in balance and ensures that the microalgae do not enter the declining growth phase or the next phase, the stationary phase.

Cultures enter stationary phase when net growth is zero, and within a matter of hours cells may undergo dramatic biochemical changes. The nature of the changes depends upon the growth limiting factor. Nitrogen limitation may result in the reduction in protein content and relative or absolute changes in lipid and carbohydrate content. Light limitation will result in increasing pigment content of most species and shifts in fatty acid composition. Light intensities that were adequate or optimal for growth in the first 3 phases can now become stressful and lead to photoinhibition. It is important that while the measured light intensity within the culture will decrease with increasing biomass if the incident illumination is maintained relatively high then a large proportion of cells may become stressed, photo-inhibit and the culture can be pushed into the death phase. This is especially the case if the culture is also nutrient stressed. It is preferable for many species to halve or further reduce the incident light intensity when cultures enter stationary phase to avoid photoinhibition. Some green algae and cyanobacteria may survive in the vegetative state (i.e., not as cysts) for over 6-12 months under very low illumination. For many species lower temperature combined with lower irradiance can further reduce stress. Survival is inversely proportional to temperature but only in darkness. Some algal species may form long lived cysts or temporary resting cysts with greatly reduced metabolism under different conditions of stress. The shut-down of many biochemical pathways as stationary phase proceeds means that the longer the cells are held in this condition the longer the lag phase will be when cells are returned to good growth conditions.

The final growth phase of algae is the death phase. When vegetative cell metabolism can no longer be maintained the death phase of a culture is generally very rapid, hence the term “culture crash” is often used. The steepness of the decline is often more marked than that represented in the accompanying growth figure. Cultures of some species will lose their pigmentation and appear washed out or cloudy, whereas cells of other species may but the culture color will be maintained. The latter is an important consideration and one reason why color should not be relied upon to gauge culture health. Bacteria which may have been kept in check during exponential and early stationary phase may “explode” as cell membrane integrity become progressively compromised or leaky and a rich carbon source for bacterial growth is released. Free pigment and bacterial growth are further reasons why measures of turbidity or fluorescence should not be used beyond early stationary phase as surrogate biomass indicators, or especially as indicators of culture health. Occasionally cell growth of some species can reoccur after a culture has apparently died. In this instance most vegetative cells will have died, and possibly most of the bacteria, releasing nutrients back into the media. Then either the very few remaining vegetative cells or more likely germination of cysts or temporary cysts will be able to fund this secondary growth.

Light limitation at high biomass occurs when the cells absorb most of the incoming irradiation and individual cells shade each other (hence the often-quoted term “self-shading”). Growth in most phytoplankton is saturated at relatively low irradiances of 50-200 μmol. photons m-2 s-1 (cf. noontime irradiance at the water surface in the tropics of 2000 μmol. photons m-2 s-1). Light limitation is avoided by agitating and circulating the biomass, as detailed further below.

High levels of productivity (in terms of biomass and lipids) are crucial for viable biofuel production and can be achieved by increasing algal diversity and assembling communities based on species' eco-physiological traits. Lipids produced by microalgae can be divided into two main groups: polar lipids, like glycerophospholipids, which have an important role in cell structure; and nonpolar lipids, like triacylglycerols, mainly responsible for energy storage. During the process of photosynthesis, nonpolar lipids like triacylglycerol (TAG), end up being stored in the microalgal cells. It has been widely accepted that the production of these lipids serves as energy storage to microalgae cells. These are valuable compounds since have an important commercial value. Through the process of trans esterification, the TAGs can be easily converted into fatty acid methyl esters which are an important and versatile form of biodiesel and the cornerstone for its production, as reported by Alishah Aratboni et al. in Microbio Cell Fact (2019).

Ambient CO2 106 and ambient O2 107 are added to a biomass system through automated and manual control of pipe assembly 204, to be detailed more below. Careful management of these inputs increases the efficiency of the system and increases overall yield. Ambient CO2 106 is used in photo-autotrophic growth and ambient O2 107 is used in heterotrophic growth.

Solar energy 108 is crucial for photoautotrophic growth 102, and sunlight exposure is aided by the agitation and movement of the biomass by pipe assembly 204, to be detailed more below. The ability to expose all of the biomass to solar energy 108 enables the cultivation to take place in deeper containers, thus increasing volume of biomass cultivated and product yielded.

Organic carbon and nutrients 109 are utilized in and regenerated by both photo-autotrophic growth 102 and heterotrophic growth 103. Organic carbon and nutrients 109 are added by pipe assembly 204 and mobile overhead harvesting machinery 601, to be detailed more later.

In some exemplary embodiments of the use of mixotrophy for increased efficiency and yield, species of microalgae perform photoautotrophic growth 102 and heterotrophic growth 103 in the same local environment, collectively performing mixotrophic growth 101. Mixotrophic growth 101 reduces lost biomass from photo-inhibition, protects from photo-oxidative damage from accumulated O2, reduces nightly biomass loss, and increases lipid production with prolonged exponential growth phases.

In some exemplary embodiments of the cultivation of algae, considerations used for the selection of algae species include, but are not limited to, biochemistry, genomics, structure, and interspecies interactions.

In some exemplary embodiments of methods of cultivation of biomass, microalgae species able to perform photoautotrophic growth 102 and heterotrophic growth 103 in the same local environment, collectively performing mixotrophic growth 101 include, but are not limited to, Chlamydomonas reinhardtii, Crypthecodinium cohnii, Haematococcus pluvialis, Chlorella ssp., Micractinium inermum, Nannochloropsis oculate, Botryococcus braunii, Scenedesmus dimorphus, Thalassiosira pseudonana, Phaeodactylum tricornutum, Dunaliella salina, Tetraselmis ssp., Asterarcys quadricellulare.

Other exemplary embodiments of methods of cultivation of biomass are not limited to the previously described species; any species or combination of species that can perform photoautotrophic growth 102 and heterotrophic growth 103 in the same local environment, collectively performing mixotrophic growth 101, are part of an embodiment of this invention.

### 1B. A Scalable System for Algae Pools

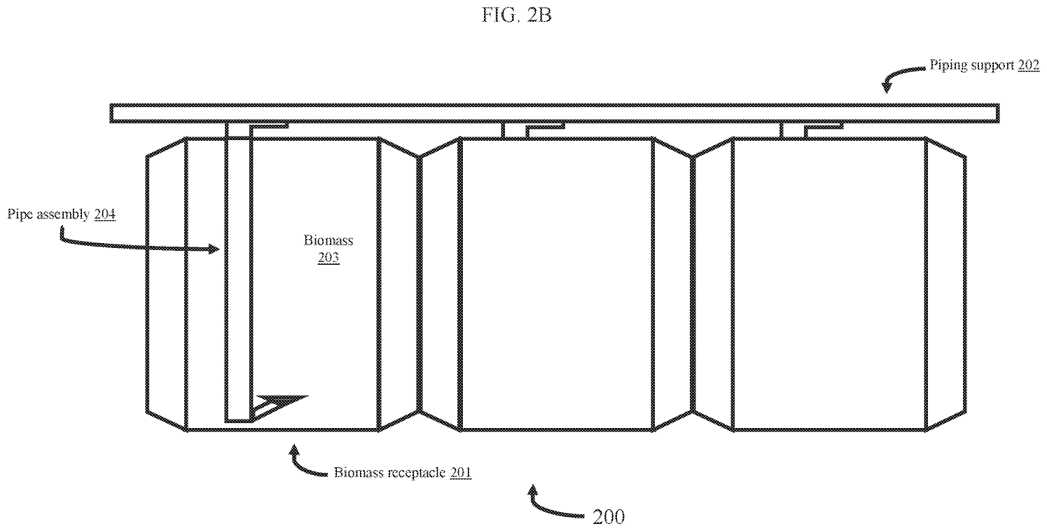

Referring now to FIG. 2A, a diagram illustrating a system for efficient biomass cultivation using a conglomerate of discrete receptacles 200 is illustrated, in accordance with some embodiments of the present disclosure. FIG. 2 only displays one section of a larger system of biomass receptacles 201.

One exemplary embodiment of a system for efficient biomass cultivation using a conglomerate of segregated pools uses 120,000 tanks on 120 acres of land. This is a modular design that enables smaller or larger numbers of biomass receptacles 201 to be used in a system. Honeycombed biomass receptacles 201 are one part of the larger HAPP (Honeycomb Algae Pool Plantation) system. Honeycombed biomass receptacles 201 are discrete environments in which each biomass receptacle 201 is unaffected by changes in other pools. This reduces contamination risk. Certain aspects of algae pond contamination are discussed in “Contamination Management in Low-Cost Open Algae Ponds for Biofuels Production. Robert C. McBride, Salvador Lopez, Chris Meenach, Mike Burnett, Philip A. Lee, Fiona Nohilly, and Craig Behnke (2014), the entire contents of which are incorporated herein by reference for all purposes. Previous methods for biomass cultivation utilize much larger and unseparated ponds/pools. If just one part of a pool is contaminated, the entire pool is contaminated. Open ponds are an excellent habitat for a plurality of algae contaminants that can act as competitors (other algae, bacteria), parasites (virus, fungi, protozoans), or consumers (protozoans, aquatic invertebrates). A number of strategies have been previously deployed to mitigate the impact of this contamination; the most effective previous approach actively deployed in the commercial production of algae using open pond technology involves maintaining an extreme culture environment, such as high salinity, high alkalinity, or high nutritional status. Other strategies have been suggested and attempted for managing contamination in open ponds. Many involve deploying a chemical into the pond that alters the pond environment to confer a differential advantage on the target algae strain, or to disadvantage the undesirable contaminant. Examples of these strategies involve the use of hyperchlorite in Nannochloropsis cultures to control protozoa and disadvantage invasive strains. Ammonia has been used as a treatment for rotifers and cladocerans in open ponds, and pH adjustments (decrease to pH 3 for 1-2 hours, followed by adjustment to pH 7.5) have been used to target rotifers. Glyphosate and ozone have been used to maintain dominance in Nannochloropsis cultures. The use of pesticides such as Dipterex, Parathion, and dichlorodiphenyltrichloroethane (DDT) to control zooplankton has been tested in laboratory-scale Chlorella sp. cultures but has not been used in large-scale cultures. None of the above chemical strategies has been successful at commercial scale or for extended periods of time in open ponds. Managing contamination in open ponds is about creating an environment in the pond that promotes the growth of the target strain(s) of algae over the growth of contaminants that may negatively impact the growth of the target strain(s). There will always be contaminants in the pond, and successful strategies will manipulate the pond environment (composition of the media, type of strains deployed, chemicals deployed, etc.) in a way that maximizes the growth rate of the target strain while simultaneously minimizing the growth rate of the contaminant(s). The invention presently disclosed achieves this goal using strategies to be detailed below.

In previous solutions relying on chemical or other similar strategies for pest management, the possibility that a target pest may evolve resistance to the pesticide should also be taken into account when considering this approach to managing contamination in open ponds. This has happened in many other agricultural settings and there is no reason to believe it could not happen in open ponds of algae. One of the strategies deployed to mitigate the risk of this happening is to use the pesticide discriminately. The initial implementation of pesticide use in agriculture, with its pest-independent doses, has driven the resistance of pests to unprecedented levels. These strategies are much more costly and difficult to implement than the invention presented here. The method of using a large number of smaller containers successfully impedes the spread of contaminations due to the inability of contaminants to spread quickly, while still being much more cost effective.

Other issues that affect open outdoor pond systems are environmental fluctuations (i.e., light levels, nutrient ratios, and temperature), invasion pressure by undesired algal species, pathogen infections, and herbivory by invading zooplankton, all of which may negatively influence the system's overall harvestable yield. Another reason that the method for biomass cultivation presented here is an improvement on previous methods is the use of diverse assemblages of algal species. This fills in the available ecological niche space, leading not only to high productivity but also reduced invisibility by undesired strains and potentially reduced susceptibility to algal diseases. A diverse assemblage of algal species also enables mixotrophic growth 101, as detailed previously.

In some embodiments of a system for biomass cultivation includes utilizing biomass receptacles 201 made of sustainable plastics. The sustainable plastic containers are made from recycled or algae-based plastics. Recycled plastic containers are projected to have a 100+ year lifespan. The use of containers made of other materials does not depart from the spirit of the invention.

In some embodiments of the presently disclosed system, rotational molding machines are used to manufacture biomass receptacles 201 on-site. This removes the shipping issues associated with ascertaining a large number of identical containers, as well as the time delays related to shipping. Rotational molding involves a heated hollow mold which is filled with a charge or shot weight of material. It is then slowly rotated, causing the softened material to disperse and stick to the walls of the mold. In order to maintain even thickness throughout the part, the mold continues to rotate at all times during the heating phase and to avoid sagging or deformation also during the cooling phase. A plurality of rotational molding machines can be used for this process.

One embodiment of the use of rotational molding machines to manufacture biomass receptacles 201 utilizes a shuttle rotational molding machine. This kind of rotational molding machine has a low energy cost and is low in cost for the size of the product produced.

One embodiment of the use of rotational molding machines to manufacture biomass receptacles 201 utilizes a swing arm rotational molding machine. This kind of rotational molding machine is useful for manufacturing pars with long cooling cycles or a long demolding process, due to the larger number of arms as compared to other rotational molding machines.

One embodiment of the use of rotational molding machines to manufacture biomass receptacles 201 utilizes a carousel rotational molding machine. This kind of rotational molding machine has two different possible forms, fixed and independent. The fixed-arm carousel works well when identical cycle times are used for each arm. The independent-arm carousel machine is available with three or four arms that can move independently. This allows for different-size molds, with different cycle times and thickness needs.

One embodiment of the use of rotational molding machines utilizes a rock and roll rotational molding machine. This kind of machine is used to manufacture specialized long and narrow parts. The uses for these parts include, but are not limited to, construction of supports around the perimeter of a group of biomass receptacles, construction of additional supports to the piping support 202.

One embodiment of the use of rotational molding machines to manufacture biomass receptacles 201 utilizes a clamshell rotational molding machine. This kind of rotational molding machine takes up less space than equivalent shuttle and swing arm rotational molding machines. A clamshell rotational molding machine is also low in cost compared to the size of products made.

One embodiment of the use of rotational molding machines to manufacture biomass receptacles 201 utilizes a vertical rotational molding machine. This kind of rotational molding machine are energy-efficient due to the compactness of their heating and cooling chambers. A vertical rotational molding machine has similar capabilities to a carousel machine but takes up less space.

In some embodiments of the presently disclosed system, algae-based plastics are used to fabricate the walls of the biomass receptacles 201. Dry algae cake, one of the by-products of the extraction process, is ground and blended with a binding biopolymer. This mix is then further processed into a filament useable in 3D printers. Industrial 3D printers then construct biomass receptacles 201 of the appropriate size.

In some embodiments of the presently disclosed system, algae are used to recycle biomass receptacles 201 or other plastic containers. One example of when this would be utilized is if a biomass receptacle 201 fails and is unable to be used. The biomass receptacle 201 could then be recycled by the addition of a bacterial enzyme which enables the algae to break down the plastic.

The embodiments disclosed wherein provide for a piping support 202 that supports a system of pipes, which enables individual control of every biomass receptacle 201. The piping support 202 can support the piping in a plurality of embodiments.

In some embodiments of the presently disclosed invention, piping support 202 supports a pipe system by enclosing in a larger supporting structure. This protects the piping system from environmental or otherwise negative effects.

In some embodiments of the presently disclosed invention, piping support 202 supports a pipe system by an underside concave structure. This enables ease of access for maintenance or otherwise manual or automated intervention.

Biomass 203 is the cultivated algae biomass which is ultimately harvested and converted to the crude algae oil product. Prior to seeding, microalgae are maintained in a separate microalgae nursery set of pools. This nursery set of pools is used for research and development, testing of different compounds and other variables, and quality control. Once the algae has been seeded, no reseeding is required because the algae maintain their peak growth phase indefinitely, doubling their mass roughly every 24 hours.

Other embodiments of the preparation for algae could be utilized without departing from the spirit of the invention. Other methods of preparation of algae prior to seeding include, but are not limited to, seeding the biomass receptacle 201 with algae not prepared in a nursery, seeding new algae into a biomass receptacle 201 with already matured algae, growing the algae in a highly sequestered and monitored environment prior to seeding, or any other similar methods of growing algae in a closely monitored setting.

Another embodiment of the presently disclosed system is the framework for preventing system failures. Predictive analytics are used to predict system failures through a combination of time in service calculation and sensor data. Sensor data includes, but is not limited to, piezo electric vibration sensors, audio sensors, resistive temperature detectors, and moisture sensors. The primary risk of failure is the bearings in electrical motors which the harvesting equipment and air pumps. Motors are also routinely checked with a sound scope to identify potential bearing issues prior to failure. Spare bearing and motors as well as replacement parts for the major harvesting equipment components are kept on site.

One embodiment of the presently disclosed method for system failure detection through predictive analytics utilizes temporal data mining. Certain aspects of failure detection are discussed in “Predicting Time-to-Failure of Industrial Machines with Temporal Data Mining. Jean Nakamura (2007), the entire contents of which are incorporated herein by reference for all purposes. Temporal data mining is a method of choice to predict future events based on known past events. The difficulty in determining time-to-failure (TTF) of industrial machines is that the failure mode is not a linear progression. The progression of a severity of a fault increases at a higher rate as the machine approaches failure. Through experience, discrete frequencies in the vibration spectra are determined to be associated with machine faults and will reach expected amplitudes at the point of machine failure.

Machinery maintenance practices have changed greatly over time. Originally, a machine would fail (run-to-failure) before maintenance is performed. This type of maintenance is sometimes called “crisis maintenance”. Then machines with no problems had preventive maintenance performed according to some schedule improved machine uptime. Now, with predictive maintenance, early identification of machine faults results in maintenance being performed before failure. With a time-to-failure (TTF) estimate, maintenance can be scheduled at the most efficient and convenient time. The most important goal of any maintenance program is the elimination of machine breakdowns. Very often, a catastrophic breakdown will cause significant peripheral damage to the machine, greatly increasing the cost of the repair. The second goal is to anticipate and plan for maintenance needs. This enables planning for down time, ordering of parts, and scheduling appropriate staff. For example, the mobile overhead harvesting machinery harvesters 601 run only during night hours and the air pumps run only during daylight hours, giving half days for cleaning, maintenance, and repairs. The third goal is to increase plant production by reducing machine breakdowns during operations. Predicting TTF can assist in achieving these goals.

Piezo-electric vibration sensors are a key component of predicting system failure. Unwanted vibrations can lead to accelerated aging and fatigue, and thus, can be detrimental to a wide variety of structures and machines. On the other hand, vibration pattern of structures is correlated to structural changes and can be used for preventive or early maintenance. Therefore, monitoring and detecting vibrations is critical for many systems and their surrounding environment. Piezoelectric sensors work on the principle of piezoelectric effect. Piezoelectric sensors work by applying mechanical energy to a crystal in three steps. First, a piezoelectric crystal is placed between two metal plates which are normally in a perfect balance (even if they are not symmetrically arranged) and does not conduct any electric current. Second, mechanical stress or force are applied on the material by the metal plates, which forces the electric charges within the crystal out of balance. Excess negative and positive charges appear on opposite sides of the crystal face. And third, the metal plate collects these charges, which can be used to produce a voltage and send an electrical current through a circuit—transforming to piezoelectricity. Piezoelectric Sensors based on the piezoelectric effect can operate from transverse, longitudinal, or shear forces, and are insensitive to electric fields and electromagnetic radiation. The response is also very linear over wide temperature ranges, making it an ideal sensor for rugged environments. Piezoelectric sensors also offer very high frequency response that means a parameter changing very rapidly can be sensed easily. Piezoelectric sensors also have high transient response as they are able to detect the events in microseconds and also give the linear output. Piezoelectric sensors also offer a high output that can be measured in the electronic circuit. Piezoelectric sensors also have small dimensions and have rugged construction which means they are easy to handle. Piezoelectric sensors therefore create extremely important data points in predictive analytics.

Other embodiments of system failure prevention include utilizing sound scopes to identify potential bearing issues prior to failure. A sound scope is a technician's aid for detecting and locating sources of abnormal noise. A sound scope transmits and amplifies through a sensitive diaphragm, allowing technicians to quickly find and analyze the source of a problem in engines or other moving parts. A sound scope can be used to pinpoint worn or defective bearing; locate cylinders with engine knocks; and determine if any fuel injectors are hung-up or inoperative.

Other embodiments of system failure prevention include the use of a number of diagnostic tools, such as clamp-on ammeters, temperature sensors, a Megger or oscilloscope to help illuminate any problems. Preliminary tests generally are done using the ubiquitous multimeter. This tester is capable of providing diagnostic information for all kinds of motors. Another essential tool for motor current measurement is the clamp-on ammeter. It circumvents such difficulties by measuring the magnetic field associated with the current, displaying the result in a digital or analog readout calibrated in amperes. A clamp-on ammeter measures the start-up and running current for any motor while itis connected to a load. The reading can then be compared to documented or nameplate specifications. As motors age, the current drawn generally rises because winding insulation resistance drops. Excess current causes heat, which must be dissipated. Insulation degradation accelerates until there is an avalanche event, causing motor burn out. The clamp-on ammeter reading indicates where the motor being tested stands on this continuum. As part of routine motor maintenance, periodic current readings are taken and put into a log posted nearby so damaging trends can be spotted in advance to avoid expensive downtime.

Another instrument used is the insulation resistance tester (or megohmmeter), generally known by its trade name Megger, can provide critical information regarding the condition of motor insulation. Periodic tests are performed, and the results recorded so damaging trends can be detected and corrected to prevent an outage and extensive downtime. The insulation resistance tester resembles a conventional ohmmeter. But rather than the typical three-volt test voltage derived from an internal battery and present at the probes, the Megger provides a much higher voltage applied for a proscribed length of time. The leakage current through insulation, expressed as resistance, is displayed so it can be graphed. This test may take place on installed or on-the-reel cable, tools, appliances, transformers, power distribution subsystems, capacitors, motors and any type of electrical equipment or wiring. The Megger is capable of applying different voltages, and the level is coordinated with the type of equipment under test and the scope of the inquiry. The test generally applies between 100 and 5,000 V or more. A protocol is composed with considerations like voltage level, time duration, intervals between tests and connection methods, and also taking into account the type and size of the equipment, its value and role in the production process and other factors.

Other embodiments of system failure prevention include the use of newer more contemporary instruments. For instance, test equipment such as Fluke's 438-II Power Quality and Motor Analyzer uses algorithms to analyze not only three-phase power quality but also torque, efficiency, and speed to determine system performance and detect overloaded conditions, eliminating the need for motor load sensors. The 438-II provides analysis data for both the electrical and mechanical characteristics of the motor while in operation. The 438-II also measures the three-phase current and voltage waveforms and uses learning algorithms to compare them against rated specifications to calculate motor mechanical performance. The analysis is presented in simple readouts, making it easy to gauge the operating performance and determine if adjustments are needed before failures cause an operational shut down. The analyzer also provides measurements to determine a motor's efficiency (for example, the conversion of electrical energy to mechanical torque) and mechanical power under operating load conditions. These measures allow for determining the motor's in-service operating power compared to its rated power to see if the motor is operating in overloaded condition or, conversely, if it's oversized for the application, energy may be wasted, and operating cost increased. Other contemporary instruments possibly used include integrating multiple instrument functions into one unit. For instance, a new thermal imaging clamp-on ammeter from FLIR has a built-in infrared camera, which gives the user a visual indication of temperature differences and thermal anomalies.

Another embodiment of preventing system failures is inherent redundancies in the HAPP system that reduce risks and impacts of failures. For example, many thousands of pools versus one large pond. If one large pond is being used, and there is a puncture or leakage in the container, it is very possible that large amounts of biomass will be lost, if not all of the biomass. Using thousands of pools means that even if several pools were punctured, the consequences would be much less dire. Only a small amount of biomass would be lost and every other container would be unaffected. As detailed above, this similar principle applies to contamination.

Another embodiment of inherent redundancies is the use of dozens of individual harvesting baskets versus one of two straining systems normally used in ponds. Straining systems are inherently prone to be clogged. the selection of a method for microalgal straining or filtration is a difficult task because it must take into account microalgal cells' properties, such as morphology, density, and size, and final product specifications. Having a large number of strainers compensates for the difficulties associated with selecting an aperture size that works for every kind of algae and environment. If there is only one or two straining centers, the aperture size must always be perfect, lest there be large-scale recurrent problems.

Another embodiment of inherent redundancies is the use of one air pumping system per 100 pools versus one centralized high-pressure system normally used. One air pumping system per 100 pools means that only one section of a system will fail if an air pumping system fails. This also lowers the pressure exerted on the air pumping system. When one centralized high-pressure air system is used, not only are system failures more likely due to the higher pressure on the system, but the system failures are also much more devastating to productivity because all of the pools will be affected. Without an air pump system, it is much more difficult to control the biomass environment and ensure the highest productivity. Having a larger number of smaller air pump systems is therefore an important improvement over one central high-pressure air system.

Another embodiment of the presently disclosed system is the use of electric water pumps that use on-site generation. This means that the electric water pumps are fueled by algae crude oil, which is manufactured from the facility. This enhances sustainability and decreases overall costs. There is also a grid connection used as a back-up. Solar water heaters used as the primary source of hot water during the extraction cycle, but when secondary hot water is required, water heaters powered by crude algae oil produced-on site are used.

Another embodiment of the presently disclosed system is the use of electric water pumps that use solar power. This is another method of enhancing sustainability while decreasing overall costs.

Another embodiment of the presently disclosed method is the use of seawater to fill up the biomass receptacles 201. Seawater is a cheap source of water, reducing overall costs. In some embodiments, some water is removed during harvesting and is then easily replenished by seawater. The use of other kinds of water, such as pure fresh water, distilled water, or other kinds of water habitable to algae, would not depart from the spirit of the invention.

Referring now to FIG. 2B, a diagram of a cross-section view of an exemplary system for monitoring and controlling individual biomass receptacles 200 is illustrated, in accordance with some embodiments of the present disclosure. Being able to monitor and control individual biomass receptacle 201 settings enables for precise adjustments and interventions, increasing yield.

A pipe assembly 204 is within each of the biomass receptacles 201 in an overall system for biomass cultivation 100. Biomass receptacles 201 are not pierced by a pipe assembly 204, lowering the chance of leakage and cost of maintenance. The pipe assembly 204 reaches the bottom of the biomass receptacles 201, enabling the most effective circulating and agitating approach. The pipe assemblies 204 are able to affect the environment of the biomass 203 by dispensing various chemicals, to be detailed further below. The pipe assemblies 204 are also able to monitor the environment of the biomass 203 using a number of sensors present in the pipe assembly.

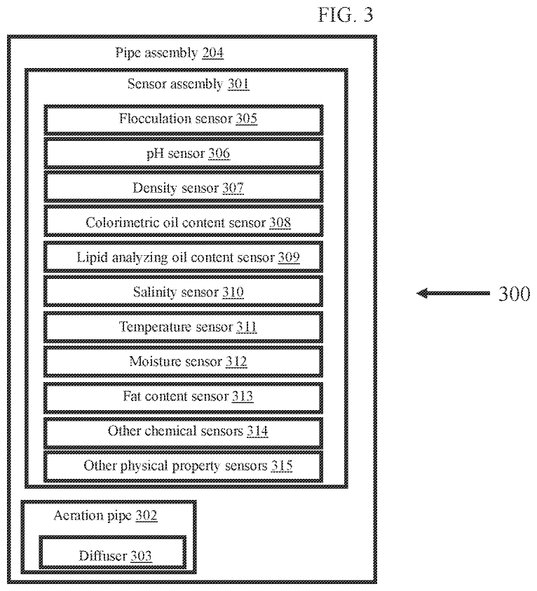

Referring now to FIG. 3, a block diagram of the components of pipe assembly 204 is illustrated, in accordance with some embodiments of the present disclosure. Pipe assembly 204 is present in each biomass receptacle 201.

Sensor assembly 301 contains a list of sensors which may include, but are not limited to: flocculation sensor 305, pH sensor 306, density sensor 307, colorimetric oil content sensor 308, lipid analyzing oil content sensor 309, salinity sensor 310. Temperature sensor 311, moisture sensor 312, fat content sensor 313, other chemical sensors 314, and other physical property sensors 315. Details of these sensors and controls affecting them are elaborated further below.

Aeration pipe 302 specifically dispenses both CO2 and O2 through the diffuser 303. This is different than most other biomass cultivation systems, which use only CO2. The aeration pipe 302 also agitates and circulates the biomass 203 within each biomass receptacle 201. This allows for increased sunlight exposure, which is critical for photoautotrophic growth 801. This also allows the biomass receptacles 201 to be deeper without having concern about lack of sunlight exposure. In previous solutions to biomass cultivation difficulty with sunlight exposure has limited the depth of biomass pools, ponds, or other similar containers. The size of the orifice on the diffuser 303 is also adjustable, changing the amount of air, pressure, and number of hours that the algae are exposed to the CO2 and/or O2.

Another embodiment of a method for agitation and circulation of biomass environment is the use of stirring mechanisms. A plurality of stirring mechanisms can be used, including, but not limited to, mechanical, magnetic stirring, current based stirring, or a combination of technologies.

One embodiment of a stirring mechanism is magnetic stirring. Magnetic stirring employs a rotating magnetic field to cause a stir bar immersed in a liquid to spin very quickly, thus stirring it. The rotating field may be created either by a rotating magnet or a set of stationary electromagnets, placed beneath the biomass receptacle 201.

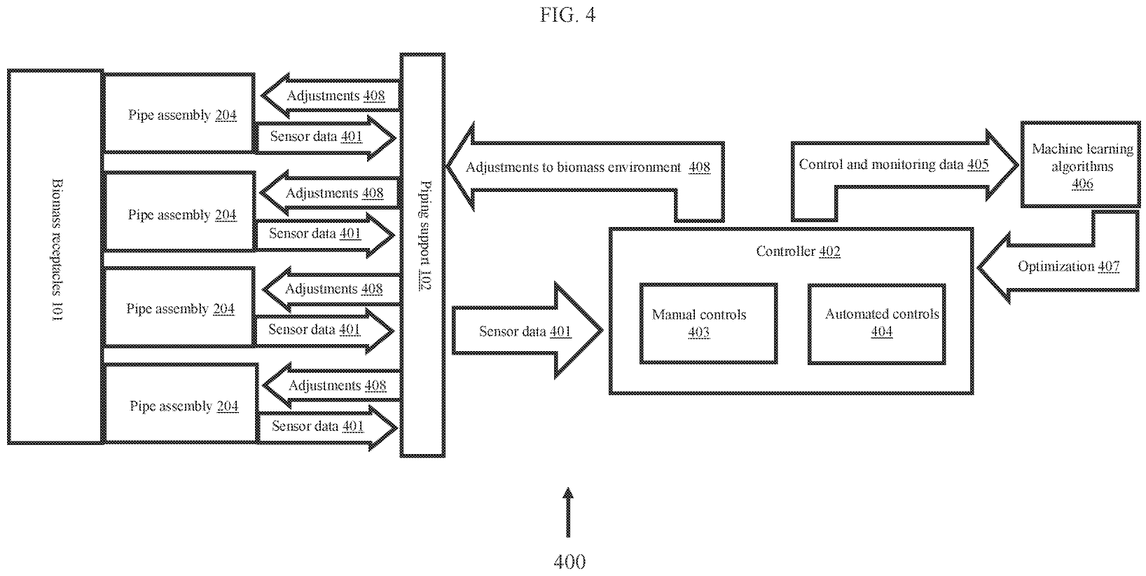

Referring now to FIG. 4, a block diagram of exemplary embodiment of the process of maintenance and optimization of biomass cultivation 400 is illustrated, in accordance with some embodiments of the present disclosure. Using data collected by various sensors in sensor assembly 301, various adjustments to a biomass receptacle or biomass receptacles 201 are affected.

In some embodiments of a system for maintenance of biomass cultivation, sensor assembly 301 may use wireless connectivity to transmit data to controller 402.

In some embodiments of a system for maintenance of biomass cultivation, sensor assembly 301 may use directly wired connections to transmit data to controller 402.

In some embodiments of a system for maintenance of biomass cultivation, any number of properties of a specifically desired individual or group of biomass receptacles 201 can be recalled by using the contents of the corresponding pipe assembly 204, which is carried by piping support 202 over large groupings of biomass receptacles 201. Biomass receptacle 201 can be monitored via automated controls 404 to administer adjustments to biomass environment 408. Certain embodiments of some of the monitorable properties are detailed below.

In some embodiments of a system for maintenance of biomass cultivation, any specifically desired individual or group of biomass receptacles 201 can have a number of properties adjusted by using the contents of the associated pipe assembly or assemblies 201, which is carried by piping support 202 over large groupings of biomass receptacles 201.

In some embodiments of a system for maintenance of biomass cultivation biomass receptacle 201 can be affected via manual controls 403 of controller 402 or via manual intervention of biomass receptacle 201 to administer adjustments to biomass environment 408. Certain embodiments of some of the adjustable properties are detailed below.

In some embodiments of a system for maintenance of biomass cultivation, adjustments to a biomass receptacle 201 can undergo optimization 407 using machine learning algorithms 406 given the input of control and monitoring data 405.

In some exemplary embodiments of a method for maintenance of biomass cultivation, the salinity of a biomass receptacle 201 can be monitored via salinity sensor 310 within sensor assembly 301, manual measurements, or other methods of calculating salinity in biomass receptacles.

In some exemplary embodiments of a method for maintenance of biomass cultivation, the salinity of a biomass receptacle 201 can be affected via manual controls 403 of controller 402, automated controls 404, or manual intervention of biomass receptacles to administer adjustments to biomass environment 408. Adjustments to biomass environment salinity may include, but may not be limited to, altering the amount of distilled, fresh, sea, or brackish water in the biomass receptacle, or adding a saltwater solution to the biomass receptacle.

The salinity content and methods of adjusting salinity content of a biomass receptacle 201 can undergo optimization 407 using machine learning algorithms 406 given the input of control and monitoring data 405.

In some exemplary embodiments of a method for maintenance of biomass cultivation, the pH of a biomass receptacle 201 can be monitored via pH sensor 306 within sensor assembly 301, manual measurements, or other methods of calculating pH in biomass receptacles.

In some exemplary embodiments of a method for maintenance of biomass cultivation, the pH of a biomass receptacle 201 can be affected via manual controls 403 of controller 402, automated controls 404, or manual intervention of biomass receptacles to administer adjustments to biomass environment 408. Adjustments to biomass environment pH may include, but may not be limited to, altering the salinity of the biomass receptacle, altering the amount of distilled, fresh, sea, or brackish water in the biomass receptacle, adding pH-increasing substances, or adding pH-decreasing substances.

The pH content and methods of adjusting pH of a biomass receptacle 201 can undergo optimization 407 using machine learning algorithms 406 given the input of control and monitoring data 405.

In some exemplary embodiments of a method for maintenance of biomass cultivation, the temperature of a biomass receptacle 201 can be monitored via temperature sensor 311 within sensor assembly 301, manual measurements, or other methods of calculating temperature in biomass receptacles.

In some exemplary embodiments of a method for maintenance of biomass cultivation, the temperature of a biomass receptacle 201 can be affected via manual controls 403 of controller 402, automated controls 404, or manual intervention of biomass receptacles to administer adjustments to biomass environment 408. Adjustments to biomass environment temperature may include, but may not be limited to, altering the amount of distilled, fresh, sea, or brackish water in the biomass receptacle, changing the location of the biomass receptacle, or using a cooling or heating element.

The temperature and methods of adjusting temperature of a biomass receptacle 201 can undergo optimization 407 using machine learning algorithms 406 given the input of control and monitoring data 405.

In some exemplary embodiments of a method for maintenance of biomass cultivation, the aeration of a biomass receptacle 201 can be monitored via aeration pipe 302, manual measurements, or other methods of calculating aeration in biomass receptacles.

In some exemplary embodiments of a method for maintenance of biomass cultivation, the aeration of a biomass receptacle 201 can be affected via manual controls 403 of controller 402, automated controls 404, or manual intervention of biomass receptacles to administer adjustments to biomass environment 408. Adjustments to biomass environment aeration may include, but may not be limited to, altering the amount of gases piped into a biomass receptacle such as oxygen or CO2.

Aeration and methods of adjusting aeration of a biomass receptacle 201 can undergo optimization 407 using machine learning algorithms 406 given the input of control and monitoring data 405.

In some exemplary embodiments of a method for maintenance of biomass cultivation, the circulation of biomass in a biomass receptacle 201 can be monitored via sensor assembly 301, manual measurements, or other methods of calculating circulation of biomass in biomass receptacles.

In some exemplary embodiments of a method for maintenance of biomass cultivation, the circulation of biomass in a biomass receptacle 201 can be affected via manual controls 403 of controller 402, automated controls 404, or manual intervention of biomass receptacles to administer adjustments to biomass environment 408. Adjustments to the circulation of biomass in biomass environment may include, but may not be limited to, altering the level of aeration in the biomass environment, altering the level of flocculation in the biomass environment, or altering the water volume in a biomass receptacle.

The oxygen content and methods of adjusting oxygen content of a biomass receptacle 201 can undergo optimization 407 using machine learning algorithms 406 given the input of control and monitoring data 405.

In some exemplary embodiments of a method for maintenance of biomass cultivation, the organic and inorganic nutrient content of a biomass receptacle 201 can be monitored via sensor assembly 301, manual measurements, or other methods of calculating organic and inorganic nutrient content in biomass receptacles.

In some exemplary embodiments of a method for maintenance of biomass cultivation, the organic and inorganic nutrient content of a biomass receptacle 201 can be affected via manual controls 403 of controller 402, automated controls 404, or manual intervention of biomass receptacles to administer adjustments to biomass environment 408. Adjustments to biomass environment organic and inorganic nutrient content may include, but may not be limited to, altering the amount of nutrients piped a biomass receptacle. Nutrients may include, but not be limited to, nitrogen or phosphorus.

Organic and inorganic nutrient content and methods of adjusting organic and inorganic nutrient content of a biomass receptacle 201 can undergo optimization 407 using machine learning algorithms 406 given the input of control and monitoring data 405.

In some exemplary embodiments of a method for maintenance of biomass cultivation, the amount of proprietary nutrient mix in a biomass receptacle 201 can be monitored via sensor assembly 301, manual measurements, or other methods of calculating the amount of proprietary nutrient mix in biomass receptacles.

In some exemplary embodiments of a method for maintenance of biomass cultivation the amount of proprietary nutrient mix in a biomass receptacle 201 can be affected via manual controls 403 of controller 402, automated controls 404, or manual intervention of biomass receptacles to administer adjustments to biomass environment 408. Adjustments to amount of proprietary nutrient mix in a biomass environment may include, but may not be limited to, altering the amount of proprietary nutrient mix piped to a biomass receptacle.

The proprietary nutrient mix and methods of adjusting a proprietary nutrient mix in a biomass receptacle 201 can undergo optimization 407 using machine learning algorithms 406 given the input of control and monitoring data 405.

In some exemplary embodiments of a method for maintenance of biomass cultivation, the carbon dioxide content of a biomass receptacle 201 can be monitored via aeration pipe 302, manual measurements, or other methods of calculating carbon dioxide content in biomass receptacles.

In some exemplary embodiments of a method for maintenance of biomass cultivation, the carbon dioxide content of a biomass receptacle 201 can be affected via manual controls 403 of controller 402, automated controls 404, or manual intervention of biomass receptacles to administer adjustments to biomass environment 408. Adjustments to biomass environment carbon dioxide content may include, but may not be limited to, altering the amount of carbon dioxide piped to a biomass receptacle.

The carbon dioxide content and methods of adjusting carbon dioxide content of a biomass receptacle 201 can undergo optimization 407 using machine learning algorithms 406 given the input of control and monitoring data 405.

In some exemplary embodiments of a method for maintenance of biomass cultivation, the density of biomass in a biomass receptacle 201 can be monitored via density sensor 307 within sensor assembly 301, manual measurements, or other methods of calculating density of biomass in biomass receptacles.

In some exemplary embodiments of a method for maintenance of biomass cultivation, the density of biomass in a biomass receptacle 201 can be affected via manual controls 403 of controller 402, automated controls 404, or manual intervention of biomass receptacles to administer adjustments to biomass environment 408. Adjustments to density of biomass in biomass environment may include, but may not be limited to, altering the amount of biomass in the receptacle, altering the species in the biomass environment, altering the aeration or flocculation in a biomass receptacle.

The density of biomass and methods of adjusting density of biomass in a biomass receptacle 201 can undergo optimization 407 using machine learning algorithms 406 given the input of control and monitoring data 405.

In some exemplary embodiments of a method for maintenance of biomass cultivation, the fat content of a biomass receptacle 201 can be monitored via fat content sensor 313 within sensor assembly 301, manual measurements, or other methods of calculating fat content in biomass receptacles.

In some exemplary embodiments of a method for maintenance of biomass cultivation, the fat content of a biomass receptacle 201 can be affected via manual controls 403 of controller 402, automated controls 404, or manual intervention of biomass receptacles to administer adjustments to biomass environment 408. Adjustments to biomass environment fat content may include, but may not be limited to, altering the amount and kind of nutrients and proprietary nutrient mix piped to biomass environment, or altering the balance of mixotrophic processes in a biomass receptacle.

The fat content and methods of adjusting fat content of a biomass receptacle 201 can undergo optimization 407 using machine learning algorithms 406 given the input of control and monitoring data 405.

In some exemplary embodiments of a method for maintenance of biomass cultivation, the moisture content of a biomass receptacle 201 can be monitored via moisture sensor 312 within sensor assembly 301, manual measurements, or other methods of calculating moisture content in biomass receptacles.

In some exemplary embodiments of a method for maintenance of biomass cultivation, the moisture content of a biomass receptacle 201 can be affected via manual controls 403 of controller 402, automated controls 404, or manual intervention of biomass receptacles to administer adjustments to biomass environment 408. Adjustments to biomass environment moisture may include, but may not be limited to, altering the seals or insulation of the biomass receptacle and piping.

Moisture content and methods of adjusting moisture content of a biomass receptacle 201 can undergo optimization 407 using machine learning algorithms 406 given the input of control and monitoring data 405.

In some exemplary embodiments of a method for maintenance of biomass cultivation, the freshwater content of a biomass receptacle 201 can be monitored via sensor assembly 301, manual measurements, or other methods of calculating freshwater content in biomass receptacles.

In some exemplary embodiments of a method for maintenance of biomass cultivation, the freshwater content of a biomass receptacle 201 can be affected via manual controls 403 of controller 402, automated controls 404, or manual intervention of biomass receptacles to administer adjustments to biomass environment 408. Adjustments to freshwater content in biomass environment may include, but may not be limited to, altering the amount of freshwater piped to the biomass receptacle.

The freshwater content and methods of adjusting freshwater content of a biomass receptacle 201 can undergo optimization 407 using machine learning algorithms 406 given the input of control and monitoring data 405.

In some exemplary embodiments of a method for maintenance of biomass cultivation, the salt water content of a biomass receptacle 201 can be monitored via sensor assembly 301, manual measurements, or other methods of calculating salt water content in biomass receptacles.

In some exemplary embodiments of a method for maintenance of biomass cultivation, the salt water content of a biomass receptacle 201 can be affected via manual controls 403 of controller 402, automated controls 404, or manual intervention of biomass receptacles to administer adjustments to biomass environment 408. Adjustments to salt water content in biomass environment may include, but may not be limited to, altering the amount of salt water piped to the biomass receptacle.

The salt water content and methods of adjusting salt water content of a biomass receptacle 201 can undergo optimization 407 using machine learning algorithms 406 given the input of control and monitoring data 405.

In some exemplary embodiments of a method for maintenance of biomass cultivation, the ratio of salt water to freshwater in a biomass receptacle 201 can be monitored via sensor assembly 301, manual measurements, or other methods of calculating the ratio of salt water to freshwater in biomass receptacles.

In some exemplary embodiments of a method for maintenance of biomass cultivation, the ratio of salt water to freshwater in a biomass receptacle 201 can be affected via manual controls 403 of controller 402, automated controls 404, or manual intervention of biomass receptacles to administer adjustments to biomass environment 408. Adjustments to ratio of salt water to freshwater in a biomass environment may include, but may not be limited to, altering the amount of either salt or fresh water piped to the biomass receptacle.

The ratio of salt water to freshwater and methods of adjusting ratio of salt water to freshwater in a biomass receptacle 201 can undergo optimization 407 using machine learning algorithms 406 given the input of control and monitoring data 405.

In some exemplary embodiments of a method for maintenance of biomass cultivation, the degree of flocculation in a biomass receptacle 201 can be monitored via flocculation sensor 305 within sensor assembly 301, manual measurements, or other methods of calculating degree of flocculation in biomass receptacles.

In some exemplary embodiments of a method for maintenance of biomass cultivation, the degree of flocculation in a biomass receptacle 201 can be affected by using manual controls 403 of controller 402 or via manual intervention of biomass receptacles to administer adjustments to biomass environment 408. Adjustments to biomass environment degree of flocculation may include, but may not be limited to, altering the mixing speed in biomass receptacles, mixing intensity, mixing time, or any other relevant properties of flocculation.

The degree of flocculation and methods of adjusting degree of flocculation of a biomass receptacle 201 can undergo optimization 407 using machine learning algorithms 406 given the input of control and monitoring data 405.

In some exemplary embodiments of a method for maintenance of biomass cultivation, the oil content of a biomass receptacle 201 can be monitored via colorimetric oil content sensor 308 or lipid analyzing oil content sensor within sensor assembly 301 transmitting sensor data 401 to controller 402, manual measurements, lipid analysis, colorimetry, or other methods of calculating oil content in biomass receptacles.

In some exemplary embodiments of a method for maintenance of biomass cultivation, the oil content of a biomass receptacle 201 can be affected via manual controls 403 of controller 402, automated controls 404, or manual intervention of biomass receptacles to administer adjustments to biomass environment 408. Adjustments to biomass environment oil content may include, but may not be limited to, altering fat content in biomass receptacle, or altering salinity in biomass receptacle.

The oil content and methods of adjusting oil content of a biomass receptacle 201 can undergo optimization 407 using machine learning algorithms 406 given the input of control and monitoring data 405.

In some exemplary embodiments of a method for maintenance of biomass cultivation, the oxygen content of a biomass receptacle 201 can be monitored via aeration pipe 302, manual measurements, or other methods of calculating oxygen content in biomass receptacles.

In some exemplary embodiments of a method for maintenance of biomass cultivation, the oxygen content of a biomass receptacle 201 can be affected via manual controls 403 of controller 402, automated controls 404, or manual intervention of biomass receptacles to administer adjustments to biomass environment 408. Adjustments to biomass environment oxygen content may include, but may not be limited to, altering the amount of oxygen piped to a biomass receptacle.

The oxygen content and methods of adjusting oxygen content of a biomass receptacle 201 can undergo optimization 407 using machine learning algorithms 406 given the input of control and monitoring data 405.

In some exemplary embodiments of a method for maintenance of biomass cultivation, the chemical properties of a biomass receptacle 201 can be monitored via chemical sensors 314 within sensor assembly 301, manual measurements, or other methods of calculating chemical properties in biomass receptacles.

In some exemplary embodiments of a method for maintenance of biomass cultivation, the chemical properties of a biomass receptacle 201 can be affected via manual controls 403 of controller 402, automated controls 404, or manual intervention of biomass receptacles to administer adjustments to biomass environment 408.

The chemical properties and methods of adjusting chemical properties of a biomass receptacle 201 can undergo optimization 407 using machine learning algorithms 406 given the input of control and monitoring data 405.

In some exemplary embodiments of a method for maintenance of biomass cultivation, the algae species of a biomass receptacle 201 can be monitored via sensor assembly 301, manual measurements, or other methods of observing algae species in biomass receptacles.

In some exemplary embodiments of a method for maintenance of biomass cultivation, the algae species of a biomass receptacle 201 can be affected via manual controls 403 of controller 402, automated controls 404, or manual intervention of biomass receptacles to administer adjustments to biomass environment 408. Adjustments to biomass environment algae species content may include, but may not be limited to, adding or removing more of the same species or combination of already present species, or adding or removing different species or combinations of species.

The algae species and methods of adjusting algae species of a biomass receptacle 201 can undergo optimization 407 using machine learning algorithms 406 given the input of control and monitoring data 405.

In some exemplary embodiments of a method for maintenance of biomass cultivation, the balance of photo-autotrophic growth 801 and heterotrophic growth 103 in a biomass receptacle 201 can be monitored via aeration pipe 302, manual measurements, or other methods of determining the balance of photo-autotrophic growth 801 and heterotrophic growth 103 in a biomass receptacle.

In some exemplary embodiments of a method for maintenance of biomass cultivation, the balance of photo-autotrophic growth 801 and heterotrophic growth 103 in a biomass receptacle 201 can be affected via manual controls 403 of controller 402, automated controls 404, or manual intervention of biomass receptacles to administer adjustments to biomass environment 408. Adjustments to the balance of photo-autotrophic growth 801 and heterotrophic growth 103 in a biomass environment may include, but may not be limited to, adding or removing more of the same species or combination of already present species, altering the amount of nutrients supplied, or altering the fat content of the algae.

The balance of photo-autotrophic growth 801 and heterotrophic growth 103 in a biomass receptacle and methods of adjusting the balance of photo-autotrophic growth 801 and heterotrophic growth 103 in a biomass receptacle 201 can undergo optimization 407 using machine learning algorithms 406 given the input of control and monitoring data 405.

In some exemplary embodiments of a method for maintenance of biomass cultivation, specified contaminants in any given biomass receptacle 201 are able to be detected on-demand using sensor assembly 301, machine learning algorithms, or other manual measurements. Some specified contaminants may include, but are not limited to, contaminants that can act as competitors (other algae, bacteria), parasites (virus, fungi, protozoans), or consumers (protozoans, aquatic invertebrates).

In some exemplary embodiments of a method for maintenance of biomass cultivation, specified contaminants in any given biomass receptacle 201 are able to be selected for filtration and screening on-demand. The specified contaminants can be filtered and screened via manual controls 403 of controller 402, automated controls 404, or manual intervention of biomass receptacles to administer adjustments to biomass environment 408.

Other such embodiments including detection and adjustment of other properties and factors do not depart from the spirit of this invention.

Other exemplary embodiments of a method for maintenance of biomass cultivation, a plurality of sensor apparatuses can be interconnected between cultivation cells. This can be done using wireless or wired technology and enables for efficient transfer of information to controller 402.

### 1C. Lighting Optimization

In some embodiments of an efficient system for the cultivation of biomass, the lighting used in the process of cultivation is optimized based on different environmental factors. Optimizations of light exposure to biomass can include, but are not limited to, pool wall alterations, supplemental electronic lighting, or supplemental bioluminescent lighting.

Certain aspects related to light optimization are discussed in “Factors Affecting Microalgae Production for Biofuels and the Potentials of Chemometric Methods in Assessing and Optimizing Productivity” by Mutah Musa, Godwin A. Ayoko, Andrew Ward, Christine Rösch, Richard J. Brown and Thomas J. Rainey (2019), the entire contents of which are incorporated herein by reference for all purposes. Microalgae are an attractive source for biofuels and bulk chemicals due to their high photosynthetic efficiency (PE). However, maximum PE values, can never be realized in microalgae mass cultures exposed to direct sunlight. Agitation and movement of biomass as in the presently disclosed invention may increase the PE, but this technique alone does not achieve the absolute maximum PE. The reason is the inherent nature of light. Unlike most chemical substances, light energy cannot be dissolved in the culture medium. Therefore there will always be a steep light gradient proceeding from a high level of sunlight to virtual darkness. In microalgal cells, photosynthesis occurs in the thylakoid, the photosynthetic membrane located inside the chloroplast. For photosynthesis to occur, light must be harvested by light-capturing pigments on light-harvesting antenna complexes. Different pigments absorb at specific wavelengths of the solar spectrum and exhibit a distinctive color. Chlorophylls are green, carotenoids are yellow and orange, and phycobilins are blue and red. In addition, they play different roles in photosynthesis. Chlorophylls are the primary pigments necessary for oxygenic photosynthesis, while carotenoids and phycobilins are important accessory pigments. Chlorophylls are the only elements of the core antenna in the photosynthetic apparatus, and they are also the key pigments of the peripheral antenna accompanied by carotenoids and phycobilins. Carotenoids can protect the photosynthetic apparatus from photo-oxidation in high-light conditions, and they expand the absorption spectrum that improve slight utilization efficiency. Phycobilins are hydrophilic and function in light harvesting and transfer, and they also play a key role in the survival of the cells in low-light conditions.

Although light is important for algal growth, levels that are too low or too high will limit or inhibit the photosynthetic process. At light levels above the upper limit for algal cells, the Photo System II (PS II) complex will get damaged, resulting in photo-inhibition and a decrease in bioproductivity. In addition, when a chlorophyll molecule is excited to the triplet state and reacts with oxygen, photo-oxidation occurs. Under high-light conditions, especially at UV wavelengths, the abundantly produced molecular oxygen acts synergistically in the presence of flavins, chlorophyll, and other photosensitizing agents to produce active oxygen (e.g., singlet oxygen, superoxide radicals). This leads to photo-oxidative damage that frequently occurs within PS II and can prevent photosynthesis by their reversibly inhibiting the function of the reaction center chlorophyll (P680). In addition, such photo-damage may lead to cell death when the oxygen reacts with fatty acids to form lipid peroxides. As detailed prior, in the presently disclosed invention, the use of mixotrophic growth stymies the negative effects of excess oxygen. In comparison, light levels that are too low will limit the growth and accumulation of microalgal bioproducts.

Blue and red light are beneficial for improving the biomass productivity and physical properties important for biodiesel. This is primarily because the most abundant pigments in most species are chlorophylls that can more efficiently absorb red and blue light compared to other colors. Chlorophyll a and b are major light harvesting pigments and are sensitive to blue and red light. Blue and red light increase photosynthetic oxygen evolution, carbon fixation and nutrient uptake. Red and blue light illumination are useful for enhancing lipid production, and therefore yield.

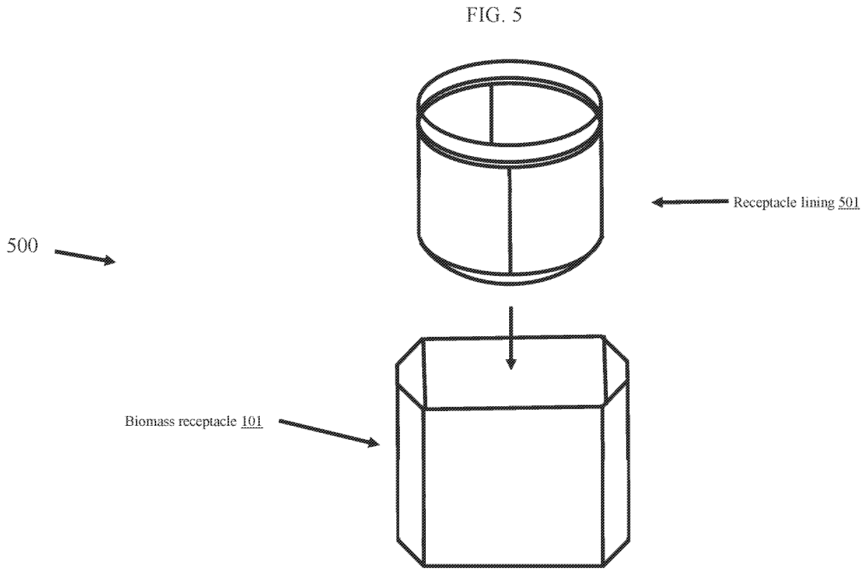

Referring now to FIG. 5, a diagram of one embodiment of a receptacle liner 501 that could be installed on the inner surface of biomass receptacle 201 is illustrated, in accordance with some embodiments of the present disclosure. The receptacle liner 501 could have a plurality of alterations to it, including, but not limited to, embedding reflective material in the receptacle liner 501 or add a reflective material coating on the receptacle liner 501. The receptacle liner 501 itself can also be made of reflective materials.

In some embodiments of the optimization of light exposure in cultivation of biomass, red and/or blue liners could be used on the inner surface of a biomass receptacle 201. This increases photoautotrophic growth and therefore product yield.

In some embodiments of the optimization of light exposure in cultivation of biomass, red and/or blue nanoparticles could be introduced to the biomass environment in a biomass receptacle 201. Nanoparticles would reflect blue and/or red light throughout the biomass environment, increasing photoautotrophic growth and therefore product yield.

In some embodiments of the optimization of light exposure in cultivation of biomass, liners allowing luminescence when the sun is not providing illumination could be used on the inner surface of a biomass receptacle 201. This increases photoautotrophic growth and therefore product yield.

In some embodiments of the optimization of light exposure in cultivation of biomass, the gene editing technology CRISPR is used for engineering algae, plasmids, or bacteria that coexist with the algae to produce bioluminescence and enable autologous growth.

The basic strategy of genome editing is to use a sequence-specific nuclease to induce a DNA double-strand break (DSB) at a target site. Thereafter, either the donor-dependent homology-directed repair (HDR) pathway or the error-prone non-homologous end joining pathway repairs the DSB and introduces some kind of genetic change. Early sequence-specific nucleases, including meganuclease3, zinc-finger nucleases4 and transcription activator-like effector nucleases5, have been shown to be effective for plant genome editing, but their construction requires complex protein engineering, which limits their applicability. CRISPR (clustered regularly interspaced short palindromic repeats)-Cas (CRISPR-associated protein) is an adaptive phage immunity system in archaea and bacteria. As they rely on DNA-RNA recognition and binding for sequence-specific nucleic acid cleavage, CRISPR-Cas9 and other CRISPR-Cas systems can be easily programmed to introduce DSBs at any desired target site at minimal cost, as reported by “The Mechanism of Double-Strand DNA Break Repair by the Nonhomologous DNA End Joining Pathway” by Michael R. Lieber (2019).

Certain aspects of gene editing are discussed in “Applications of CRISPR-Cas in agriculture and plant biotechnology” by Haocheng Zhu, Chao Li & Caixia Gao (2020), the entire contents of which are incorporated herein by reference for all purposes. Gene targeting technology in plants relies on homology-directed repair (HDR), which enables precise genome editing through the introduction of insertions, sequence replacements and nucleotide substitutions. However, the low editing efficiency achieved with HDR has limited its application in plants. Deaminase-mediated base editing and reverse transcriptase-mediated prime editing technologies are alternative genome editing technologies; as they do not involve DSB formation and do not require donor DNA, these CRISPR-Cas-based tools induce precise sequence editing and are more efficient than HDR in plants. Following the development of cytosine base editor (CBE) and adenine base editor (ABE) in human cells, dual base editor and base-editing-derived precise DNA deletion strategies were first developed in plants.

CBE is composed of a Cas9 nickase (nCas9) bearing the D10A mutation, which deactivates RuvC (one of the two Cas9 nuclease domains), fused with two proteins: a cytidine deaminase and an uracil DNA glycosylase (UDG) inhibitor (UGI). CBE introduces C:G>T:A base transitions directly into DNA sites targeted by single guide RNA (sgRNA). The deaminase deaminates cytidines to uridines in the non-target strand, which is the single-strand DNA (ssDNA) part of the R-loop generated by the nCas9 (D10A)-sgRNA complex, while the UGI prevents UDG from deaminating cytidines to apyrimidinic (AP) sites. When nCas9 (D10A) induces a nick on the target strand, the DNA mismatch repair pathway (or other DNA repair pathways) is activated and preferentially resolves the U:G mismatch into the desired U:A, and following DNA replication a T:A product, thereby generating a C:G>T:A base transition.

ABEs expand base editing to include A:T>G:C substitutions using adenosine deaminase as an effector, fused with nCas9 (D10A). Adenosine deaminase deaminates adenosines to inosines, which are recognized as guanosines by DNA polymerase during DNA repair and replication. Although there is no natural adenosine deaminase for deaminating ssDNA, such an enzyme has been evolved from Escherichia coli tRNA-specific adenosine deaminase (ecTadA). ABEs based on evolved ecTadA variants (ecTadA*) have been developed in rice, wheat, Arabidopsis thaliana and rapeseed. However, they are inefficient at some targets, and several strategies have been used to increase their editing efficiency in monocots, such as adding three SV40 nuclear localization sequences to the C terminus of nCas9, generating enhanced sgRNAs by modifying the sgRNA scaffold (scRNA) and using a simplified ecTadA* monomer version.

A cytosine and adenine dual base editor has been created to simultaneously perform C:G>T:A and A:T>G:C editing in plants using a single sgRNA19. A cytosine and adenine dual base editor uses a cytidine deaminase (APOBEC3A), an adenosine deaminase (ecTadA-ecTadA*), nCas9 (D10A) and a UGI fusion, and is referred to as ‘saturated targeted endogenous mutagenesis editor’ (STEME). The STEME system deaminates cytidines to uridine and adenosines to inosines in the editing window of the protospacer, and these are then copied by DNA repair and replication, generating dual C:G>T:A and A:T>G:C substitutions. An SpCas9-NG PAM variant, which recognizes NG PAMs, has previously been used to expand the editing scope and to increase the ability to edit as many targets as possible.

In CBEs, uridine generated by deaminating cytidines are preserved by the UGI, which inhibits the activity of the cellular UDG. The opposite situation, in which UDG is overexpressed, should trigger base excision repair and lead to excision of the uridines and generation of AP sites, which can be nicked by AP lyases. The combination of such a nick with the formation nearby of a DSB by Cas9 should produce a specified and precise deletion between the deaminated cytidine and the Cas9 cleavage site. Following this rationale, tools for generating precise multi-nucleotide deletions, which comprise a cytidine deaminase, Cas9, UDG and AP lyase—referred to as ‘APOBEC-Cas9 fusion-induced deletion systems’ (AFIDs)—have been developed to induce specific deletions within the protospacer. Two cytidine deaminases, hAPOBEC3A and the C-terminal catalytic domain of hAPOBEC3B (hAPOBEC3Bctd) have been used in AFIDs: hAPOBEC3A yields a predictable DNA deletion ranging from the targeted cytidine to the Cas9-induced DSB, and hAPOBEC3Bctd yields precise DNA deletions ranging from the TC-preference motif to the Cas9-induced DSB; these deletions ensure more uniform products. AFIDs add to the precise editing systems that facilitate the formation of in-frame deletions, interfere with DNA regulatory elements and allow editing of microRNAs.

Although CBE and ABE can induce precise base transitions, tools for generating base transversions are limited. A revolutionarily genome editing technology that solves this problem was developed in 2019. Termed ‘prime editor’, this technology can produce in human cells all 12 kinds of base substitutions, precise insertions of up to 44 bp, deletions of up to 80 bp and combinations of these edits. Prime editor uses a fusion of nCas9 (H840A) and reverse transcriptase, which is complexed with a prime editing guide RNA (pegRNA). The latter is composed of a reverse transcriptase template and a primer-binding site at the 3′ end of the sgRNA. The reverse transcriptase template contains the genetic information for the desired mutations, and the primer-binding site pairs with the nCas9 (H840A)-nicked ssDNA strand, thereby priming reverse transcription and incorporating the genetic information from the reverse transcriptase template into the genome. This is then followed by equilibration between the edited 3′ flap and the unedited 5′ flap, ligation and repair, which generate the desired edit. As prime editor generates base substitutions and short insertions and deletions at a relatively wide range of positions (+1 to +33, counting from the first base 3′ of the pegRNA-induced nick), it is not substantially constrained by its PAM.

Prime editor systems were developed and tested in rice and wheat, and were shown to generate all 12 base substitutions, multiple base substitutions simultaneously, and insertions and deletions in rice and wheat. However, the editing efficiency of prime editor in plants remains limited despite the use of orthogonal strategies, such as use of reverse transcriptase orthologues with different catalytic activities, use of ribozymes to produce precise pegRNAs, raising of the culture temperature to increase catalytic activities, incorporation of enhanced sgRNA scaffold modifications into pegRNA to increase the binding activity of Cas9 and manipulation of selective markers for enrichment of edited cells. Of note, the ability of prime editor to induce precise edits has been shown only in rice and wheat; its activity in other plants still needs to be tested. Moreover, the ability of prime editor to produce larger genetic modifications (hundreds of bases) and its specificity have not been demonstrated in either mammalian cells or plants. Thus, more work is needed to improve and expand plant prime editing technology.

Owing to its unparalleled ability to precisely manipulate plant genomes, CRISPR-Cas has emerged as a powerful tool in agriculture. It has not only helped to develop novel varieties with desirable traits but has also revolutionized current breeding systems. In addition, CRISPR-Cas has offered the possibility to domesticate novel species in a short time. Unlike conventional breeding approaches, CRISPR-Cas technology provides a rapid way to generate ideal germplasms by deleting negative genetic elements responsible for undesired traits or introducing gain-of-function mutations through precise genome editing.

Using one or a combination of the above techniques to create bioluminescent organisms would enhance the consistency of lighting, even in environments that are not sunny.

Another embodiment of light optimization is the use of supplemental man-made lighting. Electric lighting would assist in ensuring that the algae are receiving the optimal amount of light energy, especially in environments that are not sunny, or possibly an indoor setting.

One embodiment of the optimization of lighting utilizes light-emitting diodes (LEDs). LEDs are increasingly common in numerous fields due to their good cost performance, relatively high electricity-to-light energy conversion factor, varied coloration (spectra), relatively low surface temperature, long lifetime, solid-state construction without gas, etc. Plant growth and development are affected by the ambient light, including photosynthetic photon flux density (PPFD, sometimes called light intensity), cycle (light/dark period), ratio of diffuse to direct PPFD, angle determined by geometrical position or solar altitude and azimuth, and quality (wavelength or spectral distribution).

### 1D. Adjustment of Algae Metabolism

Some embodiments of a system for highly efficient biomass cultivation and high yield harvesting includes a method for the metabolism of cultivated algae such that the lipid production is optimized.

In some embodiments of a method for monitoring and adjusting the metabolism of algae the timing and concentration of added O2 and CO2 are used to optimize the metabolism of algae such that lipid production is maximized. Aeration pipe 302 within pipe assembly 204 can both detect and dispense O2 and CO2 for these purposes. Machine learning algorithms are also used in the optimization process.

In some embodiments of a method for monitoring and adjusting the metabolism of algae the sequential changes of energy production for algae are determined and used to optimize the metabolism of algae such that lipid production is maximized. Determining the sequential changes of energy production of algae can be done in a plurality of ways including, but not limited to, utilizing colorimetric oil content sensor 308, lipid analyzing oil content sensor 309, fat content sensor 313, or density sensor 307. Once the sequential changes of energy production are determined, a plurality of adjustments can be made to biomass environments. Conditions which adjustments can be applied include, but are not limited to, amount of light exposure, depth of container, volume of water, flow control and timing of nutrients, O2 and CO2, and other relevant conditions. Machine learning algorithms are also used in the optimization process.

Certain aspects of algae metabolism are discussed in “Biomass and lipid induction strategies in microalgae for biofuel production and other applications” by Hossein Alishah Aratboni, Nahid Rafiei, Raul Garcia-Granados, Abbas Alemzadeh, and José Rubén Morones-Ramírez, the entire contents of which are incorporated herein by reference for all purposes. One embodiment of affecting algae metabolism utilizes heavy metals. For example, the 56.6% increase in total lipid content in C. vulgaris at 5 different Fe3+ concentrations in the culture medium. Cadmium increases the total lipid content in Euglena gracilis. For the case of cadmium and its effects on C. vulgaris, reports have shown that TAGs, acetone mobile polar lipids (AMPL) and phospholipids (PL) were the main lipid classes after exposing C. vulgaris to different combinations of cadmium (2×10-8; 10-7 M) and nitrogen (2.9×10-6 to 1.1×10-3 M). Furthermore, by changing the combination of nitrogen and cadmium in the medium it is possible to alter and control lipid composition.

One embodiment of affecting algae metabolism utilizes types of metallic nanoparticles (NPs), within a range of 5-100 nm, since they exhibit different physical and chemical properties than the same metals at the macroscale. The diverse physicochemical behavior of metallic nanoparticles have allowed their use for many different applications in drug delivery systems, the food industry, cosmetics, optics and the synthesis of multifunctional biomaterials. One very recent application of NPs is linked to their ability to improve gas-liquid mass transfer rate in fermentations. The presence of the NPs improves the mass transfer coefficient at the gas-liquid interface; therefore, the increase of CO2 concentrations through NPs can affect the growth rate and the induction of lipids in some microalgae.

Another embodiment of utilizing nanoparticles to affect algae metabolism is the effect of silica nanoparticles and methyl-functionalized silica (SiO2-CH3) nanoparticles in a C. vulgaris culture. Using a Blue-Green medium (BG-11) and growing microalgae that used solely CO2 as a carbon source; NPs increased the gas-liquid mass transfer rate in this CO2/medium culture system and improved both growth and lipid accumulation in the cultivated microalgae. Both NPs causes an increase in the volumetric mass transfer coefficient (kLa) of 31% and 145%, respectively; although the addition of silicon NPs leads to an increase in cellular dry weight and in fatty acid methyl ester productivity, the highest cellular dry weight (1.49 g/L) and the highest fatty acid methyl ester productivity (610%) were obtained by the addition of 0.2 wt % SiO2-CH3 NPs.

Some metallic NPs such as Ag, Au, CuO, ZnO, Se, Pd and FeO turn out to be highly toxic for different organisms. One of these affected organisms is microalgae; the toxic effect of NPs is related to ROS production and the induction of oxidative stress, this is only achieved when the concentration of NPs reaches an effective level. If microalgae are exposed to adequate doses of NPs, oxidative stress can be induced and thus improve lipid production.

Another embodiment of utilizing nanoparticles to affect algae metabolism is the effects of Carbon nanotubes (CNTs), α-Fe2O3 NPs and MgO NPs on lipid production of Scenedesmus obliquus. Exposure to 5 mg/L CNTs, 5 mg/L Fe2O3 and 40 mg/L MgO NPs increases the lipid content up to 8.9%, 39.6% and 18.5%, respectively.

Another embodiment of affecting metabolism utilizes salinity levels. Salts play a vital role in the physiological and bio-chemical pathways of growth, reproduction and metabolism of fatty acids in microalgae, therefore, saline stress is one of the most efficient enrichment strategies for lipid content. Saline stress is known to cause a difference in osmotic pressure within microalgae cells, which, generates a stress-response that leads to the modification of their metabolism which will allow the microalgae to adapt to these new conditions. Changes at the metabolic level causes saline fluctuations within the cell, increasing significantly increasing the lipid content; it has even been found that variations in the concentration of salt in the growth medium not only increase the total lipids of the microalgae cells, but can also alter lipid composition.

One embodiment of utilizing salinity levels to affect algae metabolism is the effects of salt stress on the growth of marine microalgae Nannochloropsis salina. Grown at 22 PSU (particle salinity unit) until the culture reaches the stationary phase of growth, the salinity is then increased the concentration of salts to 34, 46, and 58 PSU. The lipid content increased significantly under these salt concentrations, obtaining the highest total content of fatty acids (36% dry tissue mass) at 34 PSU.

Another embodiment of affecting metabolism utilizes genetic engineering to obtain better lipid productivity. In general, genetic engineering seeks to reduce, inhibit, or over express one or several genes related to the production of a metabolite of interest. For the case of the microalgae, these genes are related with the photosynthetic process, the growth rate, improved resistance against extreme conditions such as pH, salinity, and temperature. The impact on the microalgae are related to fast growth and large cell size for high biomass production, high lipid yield, the ability to secret lipid into media, adaptive capability to environmental fluctuations and stress and the ability to form flocs for easy and low-cost harvesting.

One of the main limitations of this approach relies on the data available to do such modifications; sequencing the genomes of microalgae and having them available facilitates genetic manipulation, allowing to know with greater detail and precision the different genes that participate in the different metabolic pathways. Several nuclear microalgae genomes have been sequenced (C. reinhardtii, P. tricornutum, T. pseudonana, Cyanidioschyzon merolae, Ostreococcus lucimarinus, Ostreococcus tauri, and Micromonas pusilla). However there are a lot of ongoing projects to have more genomes available. Despite this, it is estimated that there are 72,500 species of microalgae but only about 44,000 have been described.

For genetic modification of microalgae there are a plurality of embodiments of bioengineering methods that can be applied: Random Mutagenesis, Clustered Regularly Interspaced Short Palindromic Repeats—CRISPR associated with the protein 9 (CRISPR-Cas9), Transcription Activator-Like Effector Nucleases (TALEN) and Zinc-Finger Nucleases (ZFN) used mainly for the alteration of the gene sequence; while the use of micro RNA (miRNA), short interfering RNA (siRNA) and homologous recombination allows the activation and repression of genetic expression; meanwhile agitation in the presence of glass bread or silicon, carbide whiskers, electroporation, biolistic microparticle bombardment and Agrobacterium tumefaciens-mediated gene transfer has been used to transfer DNA into microalgal cells. The efficiency of transformation strongly depends on the microalgae specie and both the genetic modification method and the transformation method must be carefully selected according to the species and type of modification.

The use of the CRISPR-Cas9 system allows the regulation of the expression of multiple target genes and the expression of complex traits through the multigene engineering. Since 2014, the use of this tool marked a beginning of a new age of genome editing in microalgae; although the main challenge of using this tool is the toxicity of the Cas9 nuclease (with a mutation rate of 10%); which has an alternative, the use of ribonucleoproteins.

The majority of the genetic editions on microalgae for the increase of lipid production have been carried out in Chlamydomonas and Chlorella. The earliest successful DNA modification was accomplished in C. reinhadtii. And in the case of fatty acid biosynthesis, acetyl-CoA carboxylase was isolated in 1990 to later transform the diatoms Cyclotella cryptica and Navicula saprophila.

One embodiment of utilizing genetic engineering to alter metabolism introduces additional copies of the acetyl-CoA carboxylase gene in the diatom Cyclotella cryptica to manipulate the lipid accumulation. Another embodiment is the transfer of the gene of a Wrinkled1 transcription factor type AP2 in Arabidopsis thaliana (AtWRI), whose main function is to regulate lipid biosynthesis in plants, into the microalgae Nannochloropsis salina. The characterization of the transformed cells revealed that the total lipid content increased by 36.5% compared to the wild-type strain.

One of the best methods in the genetic engineering approach uses the RNA silencing technique. The CrCO gene of C. reinhardtii, a homologous gene of the circadian-regulated CON-STANS gene (CO) plays an important role in the photoperiod and flowering time. The repression and overexpression of the CrCO gene can change lipid accumulation in microalgae cells and the silencing of the gene (by RNA interference, RNAi) can increase the lipid content and the levels of TAGs up to 24%. The gene knockout of a multifunctional lipase/phospholipase/acyltransferase increases the amount of lipids in the cell without affecting the growth of the T. pseudonana diatom. Antisense-mediated knockout mutants of the diatom had 3.3 times more lipid content than the wild-type variants in the exponential phase of growth.

Genetic modifications of microalgae to improve resistance could also be useful to improve lipid content/productivity. One embodiment is overexpression of the small heat shock protein (ch-sHSP) in Synechococcus elongates resulting in higher thermo tolerance under light condition (in comparison with wild-type). Another embodiment is the overexpression of HSP70B in Chlamydomonas creates greater photosynthetic efficiency by protecting the photosystem II. In addition, overexpressing the homogentisate phytyltransferase vitamin E2 (VTE2) obtains a higher protection against oxidative stress.

There are many other genetic engineering methods applied to the various species of microalgae. However, these methods have some limitations: high production cost, low growth rate, low transformation success and incomplete genetic and characterization problems for the scaling of microalgae culture.

### 1E. Nutrients and Water Delivery System

Some embodiments of a system for highly efficient biomass cultivation and high yield harvesting include a nutrient and water delivery system that makes use of wastewater as part of a proprietary nutrient mix.

Some embodiments of the presently disclosed system utilize biosolids as one component of a proprietary nutrient mix. In some embodiments, biosolids are municipal waste or sludge provided via entities that include, but are not limited to, governments disposal services or private disposal services. The biosolids provided are stabilized and stored in tanks onsite. The biosolids are mixed with water supply and fed to biomass receptacles using methods that include, but are not limited to pipe assembly 204, a nutrient delivery system built into mobile overhead harvesting machinery 601 (to be detailed further below), or other manual intervention.

Certain aspects of biosolids utilization by algae is discussed in “Microalgal Growth and Lipid Production From Organic Waste” by Zhongye Lin. (2013), the entire contents of which are incorporated herein by reference for all purposes. Biosolids similar to municipal waste or sludge contain bacteria that enhance growth of algae. Algae are capable of growing on and producing lipid from bacteria; studies of algae fermentation proved that algae can not only grow to high concentration, but produce high percentage of lipid out of their own weight as well. Other reasons that many species of microalgae are able to effectively grow in wastewater conditions is through their ability to utilize abundant organic carbon and inorganic N and P in the wastewater.

Using municipal waste is very cost effective and is acquired for only the price of shipping. In larger amounts, entities will pay to be rid of waste water. All wastewater classes contain groups of pollutants like dissolved substances (organic materials, biodegradable substances, non-biodegradable substances, inorganic materials, nutrients—these are used in part or totally by microorganisms), colloids (non-settleable small drops of oil and grease, organic and inorganic small solid particles, suspended solids, organic particles, microorganisms including bacteria, viruses, worm eggs, protozoa), mostly non-settleable other organic materials (residual particles from fruits, vegetables, meat, etc.), and mostly settleable materials (inorganic particle-sand, clay, minerals, clay, minerals). Being able to use wastewater as part of the growth medium is cost effective in and of itself, and wastewater reduces overall costs when larger amounts are used because the entities will pay to be rid of the wastewater.

Some embodiments of nutrient adjustment used in the cultivation of biomass use nutrient increases and/or deprivation to stimulate lipid production or cellulosic growth. If growth issues related to nutrition do occur, additional nutrients, organic or inorganic, may be added temporarily until the issue is remedied. A supply of inorganic nutrients is sourced from local agricultural suppliers and stored on site.

Essential inorganic nutrients such as nitrogen, sulfur, carbon, iron, and phosphorus have remarkable impact on growth, reproduction, and metabolism of microalgae cells. Nutrient limitation is an applied and promising strategy used to change and control the microalgae cell cycle and the biochemical pathways linked to lipid production and accumulation. The lack of nutrients causes undesirable situations inside the cell, generating the accumulation of more lipid compounds as a response, this technique has been therefore exploited to increase lipid production and accumulation.

In a culture medium, cell growth is linked to availability of a high concentration of nutrients in the culture, especially during the early life cycle stages of cell growth; a rich media therefore leads to a maximization of biomass productivity. Then, after reaching the necessary biomass, nutrient limitation can cause an environment of stress and a ramp-up in lipid production, especially observed in the late growth-stages. Most of the work and studies have shown that numerous species of microalgae produce and accumulate higher amount of lipids, especially the TAGs, in nitrogen-limited mixotrophic conditions.

### 2. Harvesting of Biomass

### 2A. Flocculation of Biomass

Some embodiments of a system for highly efficient harvesting of biomass utilize a method of flocculation to aid in the dewatering process. Flocculation is a chemically based separation process that requires less energy than centrifugation and ultrafiltration. Flocculation may, but is not restricted to, occur during cultivation but the benefits of flocculation are related to harvesting.

Other methods that have been previously used for enhancing microalgae harvesting include biofilm formation, filtration and screening, gravity sedimentation, flotation, and electrophoresis techniques. Of all these methods, flocculation of microalgae is shown to be the most efficient method for enhancing biomass harvesting on a large scale. Two other methods of harvesting are centrifugation and filtration.

Certain aspects of algae harvesting techniques are discussed in “Microalgae harvesting techniques: A review.” by Gulab Singh and S. K. Patidar (2018), the entire contents of which are incorporated herein by reference for all purposes. Centrifugation is an expansion of gravity sedimentation where centrifugal force replaces gravity for separating microalgae from their growth medium. Centrifugal separation of the microalgae depends on the cell settling characteristics (cell size and negligible density difference of microalgal cells to their culture medium), cell slurry retention time in the centrifuge. Centrifugation harvesting is generally characterized by high separation efficiency (>90%) under low flow rates and high energy utilization. This technique only retains viability and efficiency at laboratory scale, and becomes too costly and time consuming for industrial scale algae harvesting.

Several filter assembles have been used for harvesting algae but they are hindered by low throughput and rapid fouling. Depending on solvent/solute properties, hydrodynamic conditions, and membrane characteristics there are wide variety of filter designs. Microfiltration (MF) (0.1e10 mm), macrofiltration (10 mm), dead end filtration, ultrafiltration (UF) (0.02e0.2 mm), tangential flow filtration (TFF), vacuum filtration, and pressure filtration are a few embodiments of filtration. Typical membrane materials include polyvinylidene fluoride (PVDF), polyacrylonitrile (PAN), polyether sulfone (PES) and polytetrafluoroethylene (PTFE), polyethersulfone polyvinylpyrollidone (PES-PVP), polyvinyl chloride (PVC), an active layer of cellulose triacetate on a robust nonwoven polyester polyethylene backing (CA), ceramic filtering layers (alumina, zirconia or silica layers) on porous metal supports (cpm), and ceramic filtering layers (TiO2, Zr—O2-TiO2 or others) on porous ceramic supports (Al2O3 or ZrO2). It has been suggested that membranes with ceramic filtering layers over ceramic supports appear to be the best choice for inorganic materials. Among different evaluated organic membranes, neutral hydrophilic polyacrylonitrile (PAN) performed best in terms of permeation flux and cleanability for microalgae dewatering.

Ultrafiltration (UF) is a potential substitute for recovery but because of its high flux requirement and high operating and maintenance costs, they are generally not used for microalgae harvesting. UF membranes show better flux over a long period and fouling resistance than MF membranes, but they have very similar performance in terms of permeate flux under the same operation conditions at low transmembrane pressure (TMP). Membrane performance also depends on material and surface properties (charge, hydrophobicity). While keeping other parameters constant, positive charge membranes perform worse than their neutral counterparts. Similarly, under same operating conditions PAN (neutral and hydrophilic) performs better than PES (neutral and hydrophobic) in terms of permeate flux. Among polysulfone membrane (PS), fluoro polymer membrane (PVDF), and regenerated cellulose acetate membrane (RCA), permeate flux increased with increasing TMP or increased in the cross-flow velocity for all membranes.

Flocculation is an essential step in the concentration and harvesting of microalgae from aquatic media. Flocculation is used as an effective means of aiding the de-watering microalgae. The concentration of microalgal biomass in cultures is typically only about 0.5 to 5 g L-1, or 0.05% to 0.5%. Moreover, microalgae are small (5-20 μm) and have a density comparable to that of water. As a result, harvesting microalgae from their medium is a major challenge. The high cost of harvesting is an important reason why previous attempts to produce microalgae at large scales for low-value applications such as biofuels or bulk feed/food have failed. Most existing commercial systems use centrifugation for harvesting microalgae, but this is an energy-intensive process. Different studies showed a contribution of the costs for harvesting to more than 30% of the total cost in case of algal production in open ponds. During flocculation, the dispersed microalgal cells aggregate and form larger particles with higher sedimentation rate.

Flocculation is a cost-effective pre-concentration step for algal biomass production at industrial scale as it enables the separation of large quantities of algal biomass to be separated from the media without resulting in mechanical lysis. Conventional flocculation using chemical flocculants is costly due to high chemical (e.g., ferric sulfate, alum, polymers) dosing requirements and causes contamination of the treated wastewater and the biomass. Cationic starch and chitosan have been considered alternatives for inorganic and synthetic organic flocculants in liquid-solid separation processes in the wastewater treatment industry. However, chitosan is significantly more expensive (raw material cost 1920 cf. 400 US$) and has a more energy- and chemical-intensive preparation process than starch, and its charge is more dependent on pH condition of the applied solutions. Therefore, cationic starch is used for flocculation.

Starch is an abundant natural polymer that can be easily chemically modified by grafting different functional groups onto its backbone to enhance floc formation. Cationic starch can be prepared by addition of positively charged quaternary ammonium, amino, sulfonium, or phosphonium groups to the polysaccharide. Microalgal cells commonly carry a negative surface charge that impedes their aggregation in suspension, and so cationic starch can be used to reduce or neutralize those negative charges and aggregate the cells to improve the effective particle size and so remove them from suspension. Cationic starch is a suitable substitute for conventional flocculants to achieve effective harvesting of algal biomass due to its low cost and environmentally benign nature.

Another embodiment of algae harvesting is electroflotation. Electroflotation is a process of floating of microalgae cells to water surface with the formation of fine hydrogen bubbles by electrolysis. Hydrogen evoluted at a cathode separates microalgal biomass from the growth medium. Hydrogen bubbles sticks to the microalgal flocs and force them to float to the surface. No chemicals required and non-species specific are few benefits of this method while cathodes fouling and high-power requirements are the main disadvantages of this method.

Another embodiment of a method for algae harvesting is ozonation dispersed flotation (ODF). This method is considered as costly process for water and wastewater treatment. In this technique instead of atmospheric air, ozone gas is used to produce charged bubbles. Ozone being a strong oxidizing agent, oxidizes the soluble organic compounds and the charged bubbles separates the microalgae, thus interaction of these two processes can lead to better treatment effects. The biopolymers which are released during cell lysis acts as coagulant which is beneficial for effective separation. Ozoflotation can be applied to remove Chlorella vulgaris and Scenedesmus obliquus with turbidity removal 98% and 95%, respectively. It is also possible to achieve 79.6% TSS and 97.8% turbidity removal with 0.23 mg O3/mg dried biomass within 5 min. Furthermore, there is an increase in fatty acid methyl esters (FAME) when using ozone compared to using centrifugation. Similarly, harvesting of C. vulgaris by ozoflotation increases its lipid content by 24%. However, contamination problems may occur when used at large scale.

### 2B. Mobile Overhead Harvesting Machinery and Harvesting Basket

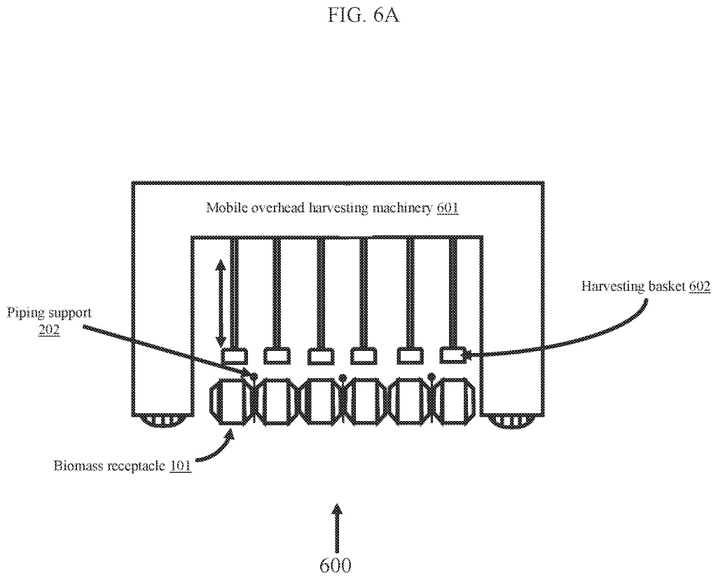

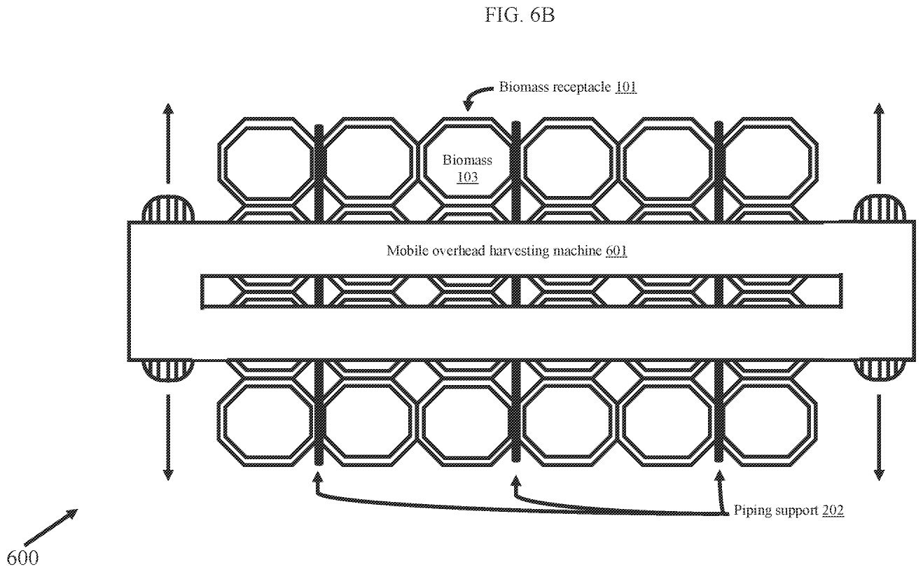

Referring now to FIG. 6A-B, a simplified diagram of a mobile overhead harvesting system 600, is illustrated, in accordance with some embodiments of the present disclosure. The exact nature of the mobile overhead harvesting machinery 601 is not especially pertinent to the spirit of the invention and the depiction of the mobile overhead harvesting machinery 601 represents that there is a system that is able to raise and lower harvesting baskets 602, or otherwise similar containers, into biomass receptacles 201, or otherwise similar receptacles.

In some exemplary embodiments of the mobile overhead harvesting machinery 601 is a built-in nutrient delivery system, as mentioned above. This system can also be utilized to make the same adjustments to the biomass environment 408 as detailed below.

In some exemplary embodiments of the mobile overhead harvesting machinery 601, the mobility of the mobile overhead harvesting machinery 601 is gained through un-tracked tires, wheels, or other similar transportation manner.

In some exemplary embodiments of the mobile overhead harvesting machinery 601, the mobility of the mobile overhead harvesting machinery 601 is gained through tracked rails alongside groupings of biomass receptacles 201.

In some exemplary embodiments of the mobile overhead harvesting machinery 601, the mobility of the mobile overhead harvesting machinery 601 is gained through a combination of technologies similar to tires or wheels and technologies similar to tracked rails.

In some exemplary embodiments of the mobile overhead harvesting machinery 601, the mobility of the mobile overhead harvesting machinery 601 is gained through permanent overhead attachments to the ceiling of warehouse containing the biomass receptacles 201 or other permanent structure.

In some exemplary embodiments of the mobile overhead harvesting machinery 601, an operator is directly present on the mobile overhead harvesting machinery 601 as it proceeds over biomass receptacles 201 or other relevant locations.

In some exemplary embodiments of the mobile overhead harvesting machinery 601, the mobile overhead harvesting machinery 601 operates automatically, without direct operator control as it proceeds over biomass receptacles 201 or other relevant locations.

In some exemplary embodiments of the mobile overhead harvesting machinery 601, the mobile overhead harvesting machinery 601 operates at night, with or without direct operator presence and control.

In some exemplary embodiments of the mobile overhead harvesting machinery 601, the mobile overhead harvesting machinery 601 operates during the day time, with or without direct operator presence and control.

In some exemplary embodiments of the mobile overhead harvesting machinery 601, the mobile overhead harvesting machinery 601 operates at both night and day with or without direct operator presence and control.

In some exemplary embodiments of the method for harvesting and cultivation of biomass, there are a variety of overhead crane types utilized in the same fashion as the previously detailed mobile overhead harvesting machinery 601. Overhead crane types include, but are not limited to, gantries, bridge cranes, monorail cranes, jib cranes, and workstation cranes.

In some exemplary embodiments of a mobile overhead harvesting system 600, the harvesting baskets 602 use a mesh screen netting, or other comparable porous material, a receptacle size dependent depth insertion and harvesting basket 602 size in such a way that only a portion of the biomass 203 in the biomass receptacle 103 is collected, and the ratio of algae to water is such that there is more algae than water harvested.

In some exemplary embodiments of a mobile overhead harvesting system 600, the harvesting baskets 602 contain sensors of the same kind as used in the pipe assembly 204. Properties able to be detected include, but are not limited to, contamination, density, and growth rate. This information can then be used to determine which tanks to harvest, either by automated controls and a machine learning algorithm or by manual operator control.

In some exemplary embodiments of a mobile overhead harvesting system 600, the harvesting baskets 602 utilize optical sensors to detect density of the growth medium, or other properties calculatable via optical sensors.

In some exemplary embodiments of a mobile overhead harvesting system 600, the parameters of biomass collection performed by the harvesting baskets 602 can be altered such that different quantities and percentages of algae biomass can be harvested from different tanks. This information can then be used to determine the best strategy for cultivating biomass, determined by either automated controls and a machine learning algorithm or by manual operator control.

In some exemplary embodiments of a mobile overhead harvesting system 600, the harvesting baskets 602 have the ability to open and close the underside of the harvesting basket 602. When lowered into a biomass receptacle 201, the underside of a harvesting basket 602 is open, and prior to be lifted out of the biomass receptacle 201, the underside closes, and a portion of the biomass 203 that was previously within a biomass receptacle 201 is retained within the harvesting basket 602.

### 2C. Overview of Entire HAPP Process

One embodiment of a method for highly efficient harvesting of biomass is the continuous harvesting of biomass. There are different phases of the HAPP process, but every phase is always happening concurrently. Different parts of the system are undergoing different phases, allowing for continuous harvesting. Continuous harvesting allows for increased yield of product.

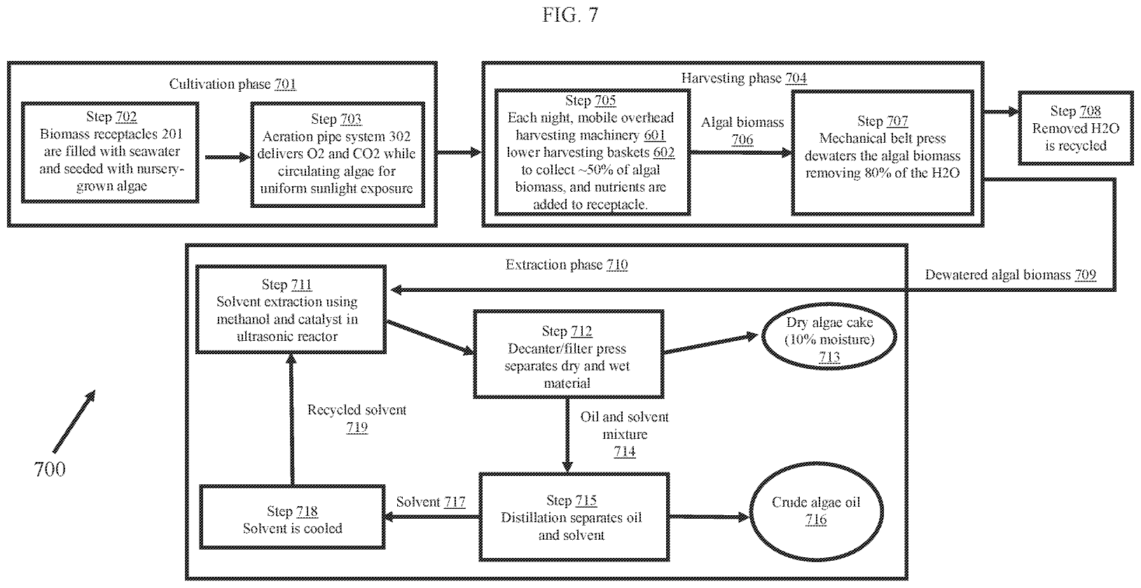

Referring now to FIG. 7, a block diagram illustrating the steps of the honeycomb algae pool plantation (HAPP) process 700, is illustrated, in accordance with some embodiments of the present disclosure. The one or more embodiments provide a system of biomass cultivation that produces over 40× higher yields than 15,000 gallon/acre-yr peak of raceway ponds. The one or more embodiments make use of various components including smaller segregated biomass containers, a large degree of automation and control over containers, mixotrophic growth, a harvesting process utilizing mobile overhead harvesting machinery, and an efficient method of extracting crude algae oil.

Cultivation phase 701 is summarized in HAPP process diagram 700 as two steps, but as detailed earlier, there are many more components than purely just this summary. Step 702 involves biomass receptacles 201 being filled with seawater and seeded with nursery-grown algae. Seawater is able to be cheaply sourced from any nearby body of water. The nursery for the algae is a separate set of containers from the main system and is used for seeding, research and development, testing, and quality control. Step 703 involves aeration pipe system 302 delivering O2 and CO2 while circulating algae for uniform sunlight exposure. Step 703 also includes monitoring and maintenance by a plurality of other sensors in sensor assembly 301 and pipe assembly 204. A plurality of adjustments to biomass environments are able to be made in a plurality of ways, as detailed above.

One embodiment of the cultivation phase 701 includes using nursery grown algae to seed biomass receptacles 201 in sequence 702, but other methods of preparation for algae could be utilized without departing from the spirit of the invention. Other methods of preparation of algae prior to seeding include, but are not limited to, seeding the biomass receptacle 201 with algae not prepared in a nursery, seeding new algae into a biomass receptacle 201 with already matured algae, growing the algae in a highly sequestered and monitored environment prior to seeding, or any other similar methods of growing algae in a closely monitored setting.

Harvesting phase 704 is the phase following cultivation phase 701, and results in algal biomass being harvested and dewatered.

Referring again to FIG. 6A, a simplified diagram of a mobile overhead harvesting system 600, is illustrated, in accordance with some embodiments of the present disclosure. One embodiment of harvesting phase 704 includes step 705 in which mobile overhead harvesting machinery 601 lower harvesting baskets 602 into one section of biomass receptacles 201 to collect a portion of the algal biomass 706 and add the proprietary organic nutrient blend to the remaining biomass 203. The mobile overhead harvesting machinery 601 then move to the next section of biomass receptacles and repeat this process.

One exemplary embodiment for a method of harvesting algae includes harvesting an average of 50% of the biomass 203 in biomass receptacles 201. Other embodiments may include harvesting a different average percent of the biomass 203 in biomass receptacle, but these do not depart from the spirit of the invention, which is harvesting only a portion of the biomass 203. Harvesting only a portion of the biomass 203 present in a biomass receptacle 201 enables the exponential growth phase of the algae to continue in the biomass receptacle 201. Once the algae have been seeded into a biomass receptacle 201, the algae are ready for harvesting in approximately 10 days, and at this point the exponential growth phase begins and the algae double in mass approximately every 24 hours. Other factors of the disclosed method for cultivation of biomass aid in the maintenance of the exponential growth phase.

Referring now to FIG. 6B, an overhead view of a simplified diagram of a mobile overhead harvesting system 600, is illustrated, in accordance with some embodiments of the present disclosure. In some embodiments of step 705, a section of biomass receptacles 201 can be taken to mean adjacent rows. There is a plurality of mobile overhead harvesting machinery sizes and the number of rows that can be harvested from simultaneously. A variation on mobile overhead harvesting machinery sizes and the number of rows that can be harvested from simultaneously does not depart from the spirit of the presently disclosed invention.

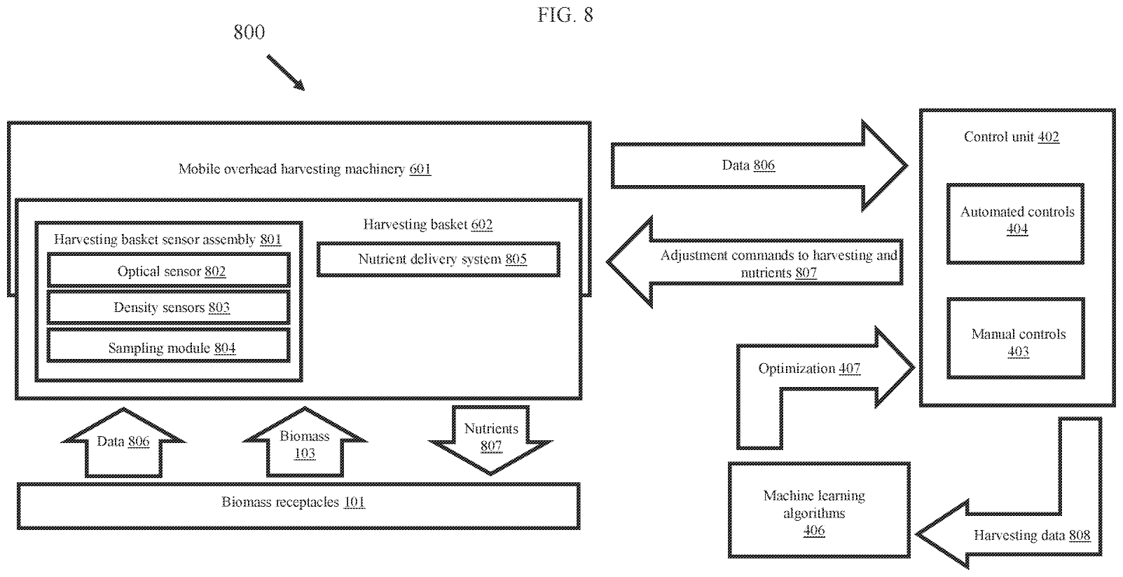

Referring now to FIG. 8, a block diagram of an exemplary system for controlling, monitoring, and harvesting biomass utilizing mobile overhead harvesting machinery 601, is illustrated, in accordance with some embodiments of the current disclosure.

In some embodiments, when a biomass receptacle 201 is harvested, automated controls 404 or manual controls 403 within controller 402 has sent a command 807 to mobile overhead harvesting machinery 601 to harvest biomass 203 from a given biomass receptacle 201. Data 806 is gathered by harvesting basket sensor assembly 801 and harvesting basket 602 harvests biomass 203 and nutrients 807 are delivered with nutrient delivery system 805. Data 806 is transmitted by mobile overhead harvesting machinery 601 back to controller 402 and added to the harvesting data 808 being sent to machine learning algorithms 406. Machine learning algorithms 406 deliver optimizations 407 to controller 402. Automated controls 404 may then be altered to include said optimizations 407.

In some embodiments, optimizations 407 may include, but are not limited to, adjustments to harvesting schedule, adjustments to which biomass receptacles 201 are being harvested from, adjustments to amount of nutrients 807 being delivered to any number of biomass receptacles 201 via nutrient delivery system 805, adjustments to the composition of nutrients 807 being delivered to any number of biomass receptacles 201 via nutrient delivery system 805, adjustments to data 806 being collected by harvesting basket sensor assembly 801, adjustments to what biomass receptacles 201 are being sampled by sampling module 804.

In some embodiments, adjustment commands to harvesting and nutrients 807 made by manual controls or automated controls may include, but are not limited to, adjustments to harvesting schedule, adjustments to which biomass receptacles 201 are being harvested from, adjustments to amount of nutrients 807 being delivered to any number of biomass receptacles 201 via nutrient delivery system 805, adjustments to the composition of nutrients 807 being delivered to any number of biomass receptacles 201 via nutrient delivery system 805, adjustments to data 806 being collected by harvesting basket sensor assembly 801, adjustments to what biomass receptacles 201 are being sampled by sampling module 804.

In some embodiments, adjustments to harvesting schedule may include, but may not be limited to, changing the time in which harvesting takes place, changing the frequency of harvesting, or changing the speed of harvesting.

In some embodiments, adjustments to which biomass receptacles 201 are being harvested may include, but not be limited to, large groups of biomass receptacles 201, individual biomass receptacles 201, what time a biomass or biomass receptacles 201 are being harvested, how frequently a biomass or biomass receptacles 201 are being harvested.

In some embodiments, adjustments to the amount of nutrients 807 being delivered to any number of biomass receptacles 201 via nutrient delivery system 805, may include, but not be limited to, increasing the amount of nutrients 807 delivered, decreasing the amount of nutrients 807 delivered, altering the frequency in which nutrients 807 are delivered.

In some embodiments, adjustments to the composition of nutrients 807 being delivered to any number of biomass receptacles 201 via nutrient delivery system 805 may include, but not be limited to, altering the amount of inorganic materials present in nutrients 807 delivered, altering the amount of organic materials present in nutrients 807 delivered, altering the amount of wastewater/biosolids included in nutrients 807 delivered.

In some embodiments, adjustments to data 806 being collected by harvesting basket sensor assembly 801 may include, but not be limited to, adjustments to which biomass receptacles 201 are being monitored, adjustments to how many biomass receptacles 201 are being monitored.

In some embodiments, adjustments to which biomass receptacles 201 are being sampled by sampling module 804 may include, but not be limited to, adjusting the number of biomass receptacles being sampled by sampling module 804, adjusting the frequency of sampling, adjusting when biomass receptacles 201 are being sampled, adjusting the volume of biomass 203 taken in a sample.

Basket sensors 801 are utilized in the determination of what adjustments to harvesting nutrients 808 need to be made to biomass receptacles 201. Harvesting basket sensor assembly 801 includes, but is not limited to, optical sensors 802, density sensors 803, sampling modules 804.

In some exemplary embodiments of a system for monitoring and harvesting biomass 203 utilizing a mobile overhead harvesting machinery 601 and harvesting basket 602, the structural integrity of a biomass receptacle 201 can be monitored via optical sensor 802 within harvesting basket sensor assembly 801, manual measurements, or other methods of calculating structural integrity in biomass receptacles.

In some exemplary embodiments of a system for monitoring and harvesting biomass 203, a mobile overhead harvesting machinery 601 and harvesting basket 602, the growth patterns of biomass 203 within biomass receptacle 201 can be monitored via optical sensor 802 within harvesting basket sensor assembly 801, manual measurements, or other methods of calculating growth patterns in biomass receptacles.

In some exemplary embodiments of a system for monitoring and harvesting biomass 203, the growth patterns of biomass 203 within biomass receptacle 201 can be affected via manual controls 403 of controller 402, automated controls 404, or manual intervention of biomass receptacles 201 to administer adjustments to harvesting and nutrients 808 utilizing harvesting basket 602 and nutrient delivery system 805. Adjustments to biomass growth patterns may include, but may not be limited to, increasing or decreasing metabolism of algae via nutrient deprivation or supplementation, reducing or increasing the amount of biomass 203 harvested from the biomass receptacle 201.

The growth patterns of biomass 203 and methods of adjusting the growth patterns of biomass 203 within a biomass receptacle 201 can undergo optimization 407 using machine learning algorithms 406 given the input of harvesting data 808.

In some exemplary embodiments of a system for monitoring and harvesting biomass 203 utilizing a mobile overhead harvesting machinery 601 and harvesting basket 602, the density of biomass 203 within biomass receptacle 201 can be monitored via mass and volume sensors within density sensors 803 within harvesting basket sensor assembly 801, manual measurements, or other methods of calculating density in biomass receptacles.

In some exemplary embodiments of a of a system for monitoring and harvesting biomass 203, the density of biomass 203 within biomass receptacle 201 can be affected via manual controls 403 of controller 402, automated controls 404, or manual intervention of biomass receptacles 201 to administer adjustments to harvesting and nutrients 808 utilizing harvesting basket 602 and nutrient delivery system 805. Adjustments to biomass density may include, but may not be limited to, increasing or decreasing metabolism of algae via nutrient deprivation or supplementation, reducing or increasing the amount of biomass 203 harvested from the biomass receptacle 201.

The growth patterns of biomass 203 and methods of adjusting the density of biomass 203 within a biomass receptacle 201 can undergo optimization 407 using machine learning algorithms 406 given the input of harvesting data 808.

In some embodiments, Step 705 of harvesting phase 704 ends when the harvested algal biomass 706 is transported to the mechanical belt press in step 707. The algal biomass 706 harvested by the mobile overhead harvesting machinery 601 is deposited by the mobile 601 onto a conveyor system or other similar kind of automated movement system. The conveyor moves the algal biomass 706 to collection hoppers, or other similar large container, and transported to the mechanical dewatering plant. Step 707 officially begins and a mechanical belt press dewaters the algal biomass, removing 80% of the water.

High energy inputs and operational cost during the dewatering of biomass are major limitations associated with microalgal biofuels development. One of the most energy and cost intensive steps in algal biomass production process is the harvesting and dewatering or drying of microalgae suspension. This is due to the low concentration in the culture medium and the microalgae small cell sizes (a few micrometers). Microalgal culture dewatering techniques commonly used are classified as chemical, mechanical, electrical, and biological. These methods can be applied as a single technique or combined.

One embodiment of algae biomass dewatering methods includes a flotation process. Flotation is a gravity separation process in which air or gas bubbles are used to carry the suspended matter to the top of a liquid surface where they can be collected by skimming process. Due to low density and self-float characteristics of some micro-algal species this method can be comparatively fast and more effective compared to sedimentation. Flotation separation has shown efficient harvesting of both fresh water as well as marine microalgae. The attachment of suspended particles to the air or gas bubbles depends on many factors including size of suspended particle, likelihood of collision and adhesion. The main advantages are short operation time, low space requirement, large scale harvesting and high flexibility with low initial cost. This process generally requires flocculants and is often proceeded by coagulation and flocculation. The surfactants increase the probability for air bubbles and suspended particles to adhere. The factors that influence the flotation efficiency include the type of collector (surfactant or flocculants), pH and ionic strength in the medium, type of bubble formation, recycling rate, air tank pressure, hydraulic retention time and particle floating rate. Micro sized bubbles are effective for separation of algal biomass from growth medium. High surface area and low-rise velocity of micro-sized bubbles leads to faster attachment of the algal cells. For flotation to be successful the cell must be hydrophobic with high molecular weight, and this can be achieved through the addition of surfactants or coagulants. Flotation processes are classified according to the method of bubble size production as dissolved air flotation (DAF), dispersed air flotation (DiAF), electrolytic flotation and ozonation-dispersed flotation (ODF).

Dissolved air flotation (DAF) uses small bubbles of sizes ranging from 10 to 100 mm, generated when air is dissolved in water under very high pressure. The bubbles force the suspended algae cells to float to the surface which later can be skimmed off, making it an energy intensive process. However, oversized bubbles can break up the floc. Surface modified bubbles can be used for the treatment of algae using DAF with 60%, 63%, and 95% removal efficiency for metal coagulant (aluminium sulphate), cationic surfactant (CTAB) and cationic polymer (PolyDADMAC) respectively. Ballasted Dissolved Air Flotation (BDAF) technique is a more consistent and sustainable harvesting system than DAF with 99% cells recovery and reduction of 80% and 95% in energy inputs and coagulant demand, respectively. There is a high energy requirement of 7.6 kWh/m3 for DAF.

In dispersed air flotation (DiAF) bubbles of 700e1500 mm size are produced by continuously passing air through a porous material (diffusers or spargers) or through a high-speed mechanical agitator. This system consumes less energy, but requires costly equipment and high pressure drop for generating bubbles. Some synthetic as well as natural collectors, such as sodium dodecylsulfate (SDS), N-cetyl-N—N—N-trimethylammonium bromide (CTAB), saponin, chitosan, etc. have been used to support the flotation process. DiAF has also been used as a function of the collector type, aeration rates and the pH of algal suspension with three surfactants, such as CTAB, SDS and the non-ionic Triton X-100, for flotation of Scenedesmus quadricauda. CTAB and SDS increased the aeration rates and reduced the size of air bubbles with algal removal efficiency of 90% and 16%, respectively. However, decreasing pH values of the algal suspension increased the removal efficiency to 80% for SDS but for CTAB no improvement in removal efficiency was seen. Flotation efficiency of anionic SDS (40 mg/L) increases from 20% to 90% by using chitosan (10 mg/L). However, chitosan (10 mg/L) use with cationic CTAB reduced flotation efficiency to 10%. When saponin is used alone it was not too effective for flotation separation of algae but with the pre-flocculation using 5 mg/L of chitosan, separation efficiency of >93% microalgae cells was observed at 20 mg/L of saponin. Performing DiAF by CTAB increases removal efficiency from 65.1% to 83.1% with increase in CTAB dose from 20 to 60 mg/L. The turbidity removal reached >90% by using pre-ozonation for 30 min. Combining dispersed air flotation with foam fractionation reveals that the highest concentration factors are gained using the following variables and variable interactions: cationic CTAB, lower surfactant concentrations, and CTAB combined with high column heights consuming only 0.015 kWh/m3 energy. Microalgae harvested by foam flotation using the surfactant CTAB have high lipid content than from cells harvested by centrifugation.

Referring now to FIG. 7, once step 707 dewatering of biomass completes, step 708 includes recycling the removed water into the biomass receptacles 201. This reduces the amount of water needing to be re-entered into the system and thus improves overall efficiency. Step 707 also results in dewatered algal biomass 709, and extraction phase 710 begins.

In some embodiments, step 710 extraction phase begins with the dewatered algal biomass undergoing solvent extraction, step 711. Solvent extraction is used to begin the oil extraction process.

In some embodiments of step 711 solvent extraction, an ultrasonic reactor utilizes methanol and chloroform to begin the oil extraction process, creating a mixture of oil, solvent, and residual biomass.

Certain aspects of ultrasonication are discussed in “Ultrasonic intensification as a tool for enhanced microbial biofuel yields” by Naveena, B., Armshaw, P., & Tony Pembroke, J. (2015), the entire contents of which are incorporated herein by reference for all purposes. Ultrasonication is a branch of acoustics that can be applied to solids, liquids, and gases at frequencies above the human hearing range. Particle agitation in a liquid culture can be achieved by applying acoustic energy with frequencies ranging from 10 kHz to 20 MHz using ultrasonic probes, an ultrasonic bath, a flat plate or a tube type ultrasonicator. The process operates by converting electrical energy into physical vibration which directly influences the medium it is applied to by imparting high energy to the medium via cavitation. During the vibration process, the microbubbles present in the form of nuclei are increased in size to a maximum of about 4-300 mm in diameter and can be either stable or transient. In the case of low ultrasonic intensity, the radii of microbubbles periodically and repetitively expand and shrink causing radial oscillation within several acoustic cycles. At the point when acoustic energy has sufficient intensity, some microbubbles become unstable and when the resonant frequency of bubbles exceeds that of the ultrasonic field, the bubbles collapse within a few nanoseconds at the solid/solvent interface (>200 mm which produces microjets with a velocity>100 m s-1 and shock waves of approximately 103 MPa towards the solid surface of the substance in solution. This causes cavitation of the substance in the liquid medium, with the violent movement of fluid towards or away from the cavitational microbubbles defined as micro-convection. The convection in the ultrasonic system has two components, microturbulence or micro-convection, and shock waves. Micro-convection is a continuous oscillatory motion of liquid medium induced by radial movement of cavitation bubble and governs the growth of the nuclei while shock waves are discrete, high pressure amplitude waves emitted by the bubble which increase the nucleation rate. This micro cavitation influences the transport of fluids and solid particles within the medium and results in forces that can cause emulsification or dispersion, while the strong shockwaves and microjets generate extremely strong shear forces over those of conventional mechanical methods and are able to scatter liquid into tiny droplets or crush solid particles into fine powders.

Ultrasonication which enhances biochemical reactions is termed ultrasonic process intensification and can be differentiated into low and high intensity applications. In several biotechnology processes, both high and low intensity ultrasonic waves have been utilized depending on the objective of the sonication process. Low intensity ultrasonic (<1 W cm-2 and between 1 and 10 MHz) intensification can be considered non-destructive as it sends ultrasonic waves through a liquid medium without causing any permanent physical or chemical change in the medium or microorganisms within. The low intensity ultrasonication can also be defined in terms of acoustic pressure amplitude. When the acoustic pressure amplitude is less than the static pressure in the medium, the bubbles undergo stable, small amplitude radial motion called “stable cavitation”. The bubble motion turns transient when the acoustic pressure amplitude exceeds the static pressure in the medium. The microorganisms in the medium respond to the low energy only during the time of exposure to the ultrasonic waves and return to an equilibrium state when the ultrasonic source is removed. On the other hand, high intensity ultrasonic intensification or low frequency ultrasonication (10-1000 W cm-2 and 10-100 kHz) which generates high pressure in the medium can disrupt microbial cellular structures. This can cause the lysis of microbial cells or the formation of free radicals in chemical degradation reactions. Thus, low intensity ultrasonic intensification is quite distinct and transitory when applied periodically. The type of cavitation generated during ultrasonic process intensification depends on several parameters, including amplitude, pressure, temperature, viscosity, and concentration of the medium.

Ultrasound causes both physical and chemical changes through the process of cavitation. Chemically, highly reactive radicals can be generated from the dissociation of the entrapped vapor molecules in a cavitation bubble. Physically, cavitation can cause intense convection in a bulk medium leading to microturbulence (an intense oscillating motion of liquid with low to moderate velocities) and shock waves (high pressurized waves emitted by the bubble, with amplitudes as high as 30-50 bar). During lipid extraction from biomass, the physical effects of ultrasonication can significantly enhance the lipid yield. Microturbulence can lead to a more efficient mixing of the biomass and solvent (without induction of shear stress), while shock waves can cause rupture of the cell wall. Ultrasound can also generate intense local turbulence in the medium, pushing the extracted lipids away from the surface of the microbial cells, and thus, maintaining a constant concentration gradient for continuous diffusion of lipids from the cells.

The physical effects of ultrasonication can also enhance the transesterification process during biodiesel production. Ultrasonication generates an enormous interfacial area between the oil and alcohol due to microturbulence leading to the formation of fine emulsions. Kalva et al. in “Physical mechanism of ultrasound-assisted synthesis of biodiesel”, investigated the mechanism of the enhancement of transesterification by discriminating the physical and chemical effects of ultrasound. This was analyzed by transesterifying soybean oil using methanol and sodium hydroxide as the base catalyst with ultrasound frequency set at 20 kHz and power output of 100 W. It was reported that the enhancement of transesterification was due to the physical effect rather than a chemical effect of ultrasound, i.e., production of radical species and acceleration of the reaction by these species. In addition, it was found that the transesterification yield increased when the alcohol to oil molar ratio was increased. This was due to the effect of low intensity microturbulence generated by the cavitation bubbles in the oil, which restricted the dispersion of oil in methanol at high alcohol to oil molar ratios.

Traditionally, solvent based processes are utilized for extraction of many bioactive compounds. However, standard extraction mechanisms such as cell maceration and soxhlet extraction have limitations such as high solvent consumption, large operating cost and extended operation times which frequently result in lower yields. The application of ultrasonic intensification can allow higher yields to be generated in a shorter time, with lower energy input and without adding additional reagents to the extraction. In addition, the effects of increasing temperature on the extraction components can be avoided. Such modified extraction techniques have been developed recently for the extraction of macromolecules such as polysaccharides, proteins, terpenoids, flavonoids, carotenoids, and phenolic compounds. The ultrasonic intensification process can be effectively used to improve the extraction rate by increasing the mass transfer due to the formation of microcavities leading to higher growth and product yields. Ying et al. in “Ultrasound-assisted extraction of polysaccharides from mulberry leaves”, reported that ultrasonic-based extraction is associated with two main physical phenomena, acoustic cavitation and diffusion through the cell wall. The conditions associated with cavitation, an increase in temperature and pressures up to 100 MPa, produce very high shear energy waves and turbulence in the cavitation zone. The combination of these factors (pressure, heat, and turbulence) is used to accelerate mass transfer in the extraction process. Ultrasonic intensification also exerts a mechanical effect which leads to enhanced diffusion of solvents into the cell wall. In pure liquids, the microbubbles retain their spherical shape during the collapse, as their surroundings are uniform. However, when the microbubbles collapse near a solid surface it occurs asymmetrically and produces shock waves toward the cell wall. These waves have a strong impact on the cell surface; therefore, enhance the solvent penetration into the cell. Another effect caused by the ultrasound wave is that it can facilitate the swelling and hydration of biomass and so cause an enlargement of pores in the cell wall which can improve diffusion processes and therefore enhance mass transfer which can enhance extraction yield. Hence ultrasonic intensification can provide high extraction efficiency in a short time with less solvent consumption over other extraction techniques. As an example, Rocco et al. in “Determination of polychlorinated biphenyls in biosolids using continuous ultrasound-assisted pressurized solvent extraction and gas chromatography-mass spectrometry” reported a 73% increase in recovery of polychlorinated biphenyls from biomass generated through treatment of household wastewater with 30 min of ultrasonic intensification during the extraction process.

Ultrasonic intensification has been shown to improve lipid extraction via cell disruption with more favorable economics than other disruption methods, enhancing the extraction of lipids by 50-500% compared to traditional methods, with a 10-fold reduction in extraction time. Suganya and Renganathan in “Optimization and kinetic studies on algal oil extraction from marine macroalgae Ulva lactuca” investigated lipid extraction from the green algae Ulva lactuca using ultrasonication and found that the yield of lipid (8.49%), was maximized with a 6 min ultrasonic pre-treatment. Lipid extraction with Synechocystis aquatilis also demonstrated that ultrasonication resulted in higher yields of lipid, with 21.30% extracted compared to grinding (18.74%), osmotic shock (14.55%) and non-disruptive methods (10.17%).

Algal cell walls are typically tri-layered rigid structures with high tensile strength, hence the release of intra lipids can be blocked. Homogenization can affect the outer cell walls with shearing force but not the interior of the cell. Extraction of lipids from cells may occur by either diffusion of lipids across the cell wall, if the algal biomass is suspended in the solvent with higher selectivity and solubility (or large partition coefficient) for lipids or disruption of the cell wall with release of cell contents in the solvent. The diffusive mechanism is less efficient due to slow diffusion of lipid across the cell wall while disruptive mechanism results in faster extraction with high yield as it causes direct release of lipid due to the rupture of cell wall. Sonication can interfere with the cell interior via shock waves produced by imploding cavitation bubbles. Thus, ultrasonic intensification can be an advantageous addition for lipid extraction processes involving algae. Park et al, in “Sonication assisted homogenization system for improved lipid extraction from Chlorella vulgaris” investigated the effect of homogenization and ultrasonication in combination on lipid extraction from Chlorella vulgaris. The initial fatty acid content of C. vulgaris was 360 mg g-1 cell. Lipid recovery was found to increase when both techniques were combined compared with the use of either alone. The results of the combined processes showed 100.5, 123.9, and 152.0 mg lipid g-1 cell recovered for the 20, 40, and 60 min reaction times used, respectively. The yields were 5.3-fold, 6.6-fold, and 8.1-fold higher, respectively, than that of the control (single treatment by sonication or single treatment via homogenization). In this system, microalgal suspensions were allowed to circulate continuously between the homogenizer and ultrasonicator and it was concluded that cell walls damaged slightly by homogenization would be effectively disrupted by the subsequent ultrasonication-induced cavitation bubbles resulting in a superior extraction process. When the cell concentration was increased to 40 g·L-1, the lipid recovery yield for 1 h of sonication-assisted homogenization was increased to a very high 281.3 mg lipid g-1 cell using the chloroform-methanol solvent. This system's treatment of high cell concentrations makes it possible to enhance lipid recovery capacity based on unit time.

A study by Glacio et al. “Extraction of lipids from microalgae by ultrasound application: Prospection of the optimal extraction method”, compared methods of ultrasonic assisted oil extraction by comparing the methods developed by Bligh and Dyer in “A rapid method of total lipid extraction and purification”, Chen et al. in “Comparison of methylene chloride and chloroform for the extraction of fats from food products”, Folch et al. in “A simple method for the isolation and purification of total lipids from animal tissues”, and Hara and Radin in “Lipid extraction of tissues with a low-toxicity solvent”. Embodiments of small-scale procedures for each of these methods are detailed below. For all of the following procedures, a mass of 5 g of dried microalgae was used in each experiment. All experiments with ultrasound application were carried out in an ultrasonic bath working at 40 kHz and producing an ultrasonic intensity of 29.7 W/L or 2.68 W/m2 (Unique model 40USC—Indaiatuba, Brazil, 2.7 L, internal dimensions: 24×14×9 cm). All experiments were carried out in triplicate.

For the Bligh and Dyer method, the biomass (5 g) was mixed and homogenized with 25 mL of methanol, 12.5 mL of chloroform and 5 mL of water. The mixture was subjected to ultrasonic energy during 40 min. Chloroform (12.5 mL) and a solution of 1.5% w/v sodium sulfate (12.5 mL) was added to the mixture and sonicated for 20 min. The extraction was carried out at ambient temperature (25° C.). Yield % achieved was 52.5±2.3.

For the Chen et al. method, the biomass (5 g) was mixed and homogenized with 25 mL of methanol and was sonicated for 3 min. Dichloromethane (50 mL) was added and the mixture was sonicated for 27 min. The extraction was carried out at ambient temperature (25° C.). Yield % achieved was 10.9±1.2.

For the Folch et al. method, the biomass (5 g) was mixed and homogenized with 25 mL of methanol. The mixture was subjected to ultrasonic energy for 3 min. Chloroform (50 mL) was added and the mixture was sonicated for 27 min. The extraction was carried out at ambient temperature (25° C.) Yield % achieved was 16.1±0.8.

For the Hara and Radin method the biomass (5 g) was mixed and homogenized with 20 mL of isopropanol and sonicated for 4 min. Hexane (30 mL) was added and the mixture was sonicated for 56 min. The extraction was carried out at ambient temperature (25° C.). Yield % achieved was 2.2±0.3.

Solvents, such as chloroform and dichloromethane, may contribute to weaken the cell wall, thus contributing toward a more intense extraction of oil from the microalgae cells. The low recovery observed for the Hara and Radin method may be attributed to the use of n-hexane, which has a nonpolar character and low selectivity toward microalgal lipids.

The higher extraction capability of the Bligh and Dyer method assisted by ultrasound can be attributed to the disruption of the cells wall provided by the cavitation mechanism, which releases lipids to the solution; and to the higher selectivity of microalgal lipids toward chloroform-methanol-water system, which has a more polar nature.

The key step in extraction and recovery of lipids from microalgae is cell disruption. Sonication increased the efficiency of the extraction since it was partially responsible for cell disruption. The selection of the proper solvent system is still essential to the extraction process because it may weaken the cell wall structure facilitating cell disruption and therefore extraction of lipids. Among these methods, the Bligh and Dyer method assisted by ultrasound resulted in the highest extraction of oil (52.5% w/w).

In some embodiments of a highly efficient method for extracting oil from biomass, a chloroform and methanol oil extraction process is utilized. In some embodiments, the algal biomass weight on a plantation is made up of an average of approximately 50% oil and 50% algal cake. A chloroform and methanol oil extraction process is able to capture approximately 99.5% of oil present in the algae biomass.

In some embodiments, the next step of extraction phase 710 is step 712, in which a decanter or filter press separates the dry and wet material of the oil, solvent, and residual biomass mixture. This then creates an oil and solvent mixture 714 and dry algae cake 713.

There are different filter designs, including pressure filters and vacuum filters that can be considered in algae separation. Pressure and vacuum filters have several different designs, including plate and frame presses, pressure and vacuum belts, and rotary presses. Experimental comparisons of filter presses showed that they are more energy efficient, reliable, and reach higher algae concentrations than vacuum filters.

In some embodiments, dry algae cake 713 has a plurality of uses. Our Dry Algal Cake (DAC) is in process of being evaluated by several pulp and paper product producers in the U.S. and Asia. DAC's primary utilization is expected to be as an additive to various blends of wood pulps, replacing a portion of the expensive tree pulps. Our DAC's market value has yet to be determined, but the costs of the various tree pulps it would replace range from $400/tonne to $1200/tonne. Once in volume production with hundreds of thousands of tonnes being produced each year as a byproduct, our DAC has the potential to offset millions of trees being cut down each year, and provide a high value byproduct revenue stream. Algal cake is extremely valuable, high protein by-product of our system that will be used for the production of nutritional food additives, vitamin supplements, animal/fish feed and other valuable consumer products.

One embodiment of dry algae cake use includes incorporation into feedstock. Feedstock used are often residues such as bark, woodchips, sawdust, or crops grown specifically for use as fuel. Comparing the calorific values of wood biomass fuels and de-oiled algae cake, there is no substantive difference between the two.

Another possible use of algae cake is in nutritional supplements. Algae cake supplements the body with protein, carbohydrates, carotenoids, amino acids, vitamins, and trace minerals. It is especially advantageous for vegetarian animals and for the individuals suffering from malnutrition. Since algae stimulate the immune system, increase white blood cell count, and promote the growth of healthy colonic flora, its supplements can be ideal for improving overall health. Algal supplements are also beneficial for treating anemia, infections, fatigue, obesity, and toxicity, as reported by Raghvendar Singh and Bhatnagar, S. K. in “Evaluation of algal cake as dietary supplement” (2011).

Algal foods offer one of the few vegetarian alternatives for cobalamin (vitamin B12) in the diet. Cobalamin is not required or synthesized by higher plants, so fruits and vegetables are poor sources of vitamin B12, which explains why vitamin B12-deficiency is common among people following strict vegetarian or vegan diets. Over half of microalgal species surveyed have a metabolic requirement for B12 and contain large amounts of such.

Certain aspects of algae cake utilization are discussed in “New insights into algae factories of the future” by Laurencas Raslavic̆ius, Nerijus Striūgas, and Mantas Felneris (2018), the entire contents of which are incorporated herein by reference for all purposes. Another embodiment of a use for algae cake is the conversion to useful gaseous products by gasification. The obtained gaseous products contain hydrogen (H2), carbon monoxide (CO), carbon dioxide (CO2), methane (CH4) and other hydrocarbons. After the processing of producer gas, the clean syngas containing only H2 and CO is produced. Finally, for the transformation of synthesis gas into valuable hydrocarbons, the catalytic Fischer-Tropsch (FT) synthesis is crucial.

Another embodiment of a use for algae cake is the conversion into fuel and chemicals by means of pyrolysis, which produces solid (bio-char), liquid (aromatic compounds, hydrocarbons, amides, amines, carboxylic acids, phenols) and gaseous fractions (H2, CO, CO2, CH4). The process is accomplished at elevated 300-600° C. temperatures and according to the process condition various yield of products could be obtained. The hydrothermal treatment seems to be most suitable for the biomass with high moisture content. During the process, algal biomass is treated at the temperatures of 250-350° C. and high-pressure (5-15 MPa). The feedstock is mainly converted to the bio-oil fraction (31-45 wt %), residual solid (6-11 wt %), dissolved aqueous constituents (17-23 wt %), and gas phase products (30-41 wt %).

Another embodiment of a use for algae cake is for terrestrial plant and environment care. One form of this is algae cake as fertilizer. Fertilizer containing algae cake has been shown to improve the growth and content of valuable compounds and gave a better quality of crop yield. The algae biomass applied in agriculture as a biofertilizer or soil conditioner provides the following advantageous properties: (i) increase in soil pores, adding filamentous structure and production of adhesive substances; (ii) production and release of plant growth regulators such as hormones (auxin, gibberellin), vitamins, amino acids antibiotics, as well as siderophores; (iii) increase in water-holding capacity through their jelly structure; (iv) increase in soil organic matter content (biomass after their death and decomposition); (v) decrease in soil salinity; (vi) preventing weeds growth; (vii) increase in soil phosphate by excretion of organic acids; (viii) provision of nitrogen by biological nitrogen fixation; (ix) antagonistic effects with soil-born plant pathogens and synergistic effects with other beneficial microorganisms, as reported by Tuhy, L., Saeid, A. and Chojnacka, K. in “Encyclopedia of Marine Biotechnology, Ch. 4 Algae fertilizers” (2020).

Certain aspects of algae utilization are discussed in “Algae as nutrition, medicine and cosmetic: the forgotten history, present status and future trends” by Maryam Anis, Salman Ahmed and Muhammad Mohtasheemul Hasan (2017), the entire contents of which are incorporated herein by reference for all purposes. Another embodiment of a use for algae cake is in the cosmeceutical industry. Their medicinally active compounds have the ability to kill bacteria and fungi that destroy the skin flora and therefore act as preservative. Algal compounds having antioxidant properties help to protect from skin aging, sun-related skin damage and other photoaging problems such as melanoma, cutaneous inflammation, and skin cancer. Skin naturally possesses antioxidants to prevent cell destabilization. However, the UV exposure generates reactive oxygen species which in turn cause free radical cell damage, cell death via apoptotic or necrotic processes. These effects are clearly noticeable by the presence of skin dryness, wrinkles, and mottled pigmentation. Tyrosinase enzyme catalyzes melanin synthesis to promote skin melanisation and tanning. Algal compounds act as tyrosinase inhibitors are the potential candidates for skin whitening. Arthrospira platensis extract can repair the symptoms of skin aging, provides a tightening effect, and inhibits stria formation; while Chlorella vulgaris extract is reported to stimulates collagen synthesis in the skin, helps in tissue regeneration and reduce wrinkle formation. Algae derived polysaccharides aid skin hydration and their moisturizing effect protects skin from dryness. This helps to maintain skin appearance, elasticity, and strengthening to provide barrier against harmful environmental factors. The polysaccharides from Saccharina japonica can absorb and retain moisture more than hydroxyl acid, the commonly used skin moisturizer in clinical practice. Therefore, algal polysaccharides may be used in cosmetics as an additive. Agar and alginic acids are good hydrocolloids and emollient and used as cosmeceutical aid.

Other embodiments of algae cake use are found in a plurality of other industries. Agar, algin and carrageenan are obtained from algae. Agar is obtained from red algae such as Gracilaria, Gelidiella, Gelidium, and Pterocladia; carrageenan from Eucheuma, Gigartina and Hypnea and algin from brown algae like Ascophyllum, Cystoseira, Lallinaria, Macrocystis, Sargassum, and Turbinaria. Agar is used as a substrate for bacteriologic culture and tissue culture eukaryotic cell in research and medical facilities. Alginates obtained from the cell wall of brown algae are used in food and pharmaceutical industries in the form of stabilizers for suspension and emulsions. Xanthophyll has a large application in the coloration of cosmetic and drugs. Phycobillins, especially blue phycobilin from Arthrospira, are water soluble pigments used as colorants for cosmetic and food products. These seaweeds are used as thickening, gelling and stabilizing agents in dairy, food, confectionary, pharmaceutical, textiles, paint, paper, and varnish industries etc. Some other chemicals such as iodine, mannitol, laminarin, fucoldin are also obtained from marine algae. Carrageenans are not only used in the food but also in textile, cosmetics, and medicines.

Referring to FIG. 7, step 712 ends when the oil and solvent mixture 714 that has been separated from the dry algae cake 713. Step 715 begins with the oil and solvent mixture being distilled, separating the oil and solvent, creating the final product, crude algae oil 716.

Distillation is a common separating technology in the chemical industry. This method separates the components successively according to their different volatilities, and it is essential for the separation of liquid mixtures. Atmospheric pressure distillation, vacuum distillation, steam distillation, and some other types of distillation have been applied in bio-oil separation.

The thermal sensitivity of bio-oil limits the operating temperature of distillation. In view of the unsatisfactory results obtained by atmospheric pressure distillation, researchers have employed vacuum distillation to lower the boiling points of components, and bio-oil could thereby be separated at a low temperature. Characterization of the distilled organic fraction showed that it had a much better quality than the crude bio-oil, containing little water and fewer oxygenated compounds, and having a higher heating value.

One embodiment of distillation of bio fuel is steam distillation. Steam distillation is performed by introducing steam into the distilling vessel, to heat the bio-oil and decrease its viscosity, and finally the volatile components are expelled by the steam. A study combining steam distillation with reduced pressure distillation, “High-Efficiency Separation of Bio-Oil” by Shurong Wang (2013), bio-oil was first steam distilled to recover 14.9% of a volatile fraction. The recovered fraction was then further distilled by reduced pressure distillation to recover 16 sub-fractions. In this process, a syringol-containing fraction was separated and syringol with a purity of 92.3% was obtained.

Due to its thermal sensitivity, it is difficult to efficiently separate bio-oil by conventional distillation methods. Molecular distillation seems to offer a potential means of realizing bio-oil separation, because it has the advantages of low operating temperature, short heating time, and high separation efficiency.

One embodiment of distillation of bio fuel is molecular distillation. There are forces between molecules, which can be either repulsive or attractive depending on intermolecular spacing. When molecules are close together, the repulsive force is dominant. When molecules are not very close to each other, the forces acting between them are attractive in nature, and there should be no intermolecular forces if the distance between molecules is very large. Since the distances between gas molecules are large, the intermolecular forces are negligible, except when molecules collide with each other. The distance between collisions with another molecule is called its free path. The mean free path of an ideal gas molecule can be described by Eq. (1): λm=k2−√πTd2p, where T (° C.) is the local temperature; λm (m) refers to the mean free path; d (m) is the effective diameter of the molecule; P (Pa) is the local pressure; and k is the Boltzmann constant.

As is apparent from Eq. (1), the molecular mean free path is inversely proportional to the pressure and the square of the effective molecular diameter. Under certain conditions, that is, if the temperature and pressure are fixed, the mean free path is a function of the effective molecular diameter. A smaller molecule has a shorter mean free path than a larger molecule. Furthermore, molecular mean free path will increase with increasing temperature or decreasing pressure.

Molecules will move more rapidly when the liquid mixture is heated. Surface molecules will overcome intermolecular forces and escape as gas molecules when they obtain sufficient energy. With an increased amount of gas molecules above the liquid surface, some molecules will return to the surface. Under certain conditions, the molecular motion will achieve dynamic equilibrium, which is manifested as equilibrium on a macroscopic scale.

Traditional distillation technology separates components by differences in their boiling points. However, molecular distillation (or short-path distillation) is quite different and precisely relies on the various mean free paths of different substances. The distance between the cooling and heating surfaces is less than the mean free path for a light molecule, but greater than that for a heavy molecule. Therefore, the light molecules escaping from the heating surface can easily reach the cooling surface and be condensed. The dynamic balance is thereby broken, and the light molecules are continuously released from the liquid phase. On the contrary, the heavy molecules are not released and return to the liquid phase. In this way, the light and heavy molecules are effectively separated.

Molecular distillation technology has been widely used in the chemical, pharmaceutical, and foodstuff industries, as well as in scientific research to concentrate and purify organic chemicals. It is a feasible process for the separation of thermally unstable materials, taking into account that it only takes a few seconds to complete the separation process. Bio-oil is a complex mixture of many compounds with a wide range of boiling points. It is thermally sensitive and easily undergoes reactions such as decomposition, polymerization, and oxygenation. Additionally, most of the compounds are present in low concentrations. Molecular distillation is not limited by these unfavorable properties and is suitable for the separation of bio-oil to facilitate analysis and quantification of its constituent compounds.

In some embodiments, once the crude algae oil 716 and solvent 717 have been separated in step 715, the solvent 717 can be cooled in step 718. This recycled solvent 719 can then be reincorporated into step 711, in which it was originally used to start the extraction process. The recycling of the solvent 717 decreases waste and increases efficiency.

In some embodiments, the crude algae oil 716 created by step 715 is then stored under a nitrogen blanket. Under open air, the crude algae oil 716 remains stable for at least 3 months. Under a nitrogen blanket, stability exceeds 6 months.

Nitrogen blanketing is a process that involves applying nitrogen to storage containers or tanks that contain hazardous chemicals that could explode if they come in contact with oxygen. This process creates a protective layer of nitrogen around the substance, and replaces any humid/moist air with dry nitrogen. This prolongs the life of the chemical or product, as well as diminishing any explosive or safety hazard associated with it.

Aspects of the various embodiments can be modified, if necessary, to employ systems, circuits, and concepts of the various patents, applications, and publications to provide yet further embodiments, including those patents and applications identified herein. While some embodiments may include all of the receptacles, sensors, harvest machinery, and other structures discussed above, other embodiments may omit some of the receptacles, sensors, harvest machinery, and or other structures. Still other embodiments may employ additional ones of the receptacles, sensors, harvest machinery, and structures generally described above. Even further embodiments may omit some of the receptacles, sensors, harvest machinery, and structures described above while employing additional ones of the receptacles, sensors, harvest machinery, generally described above.

As one of skill in the art would readily appreciate, the present disclosure comprises systems, devices, and methods for system for cultivating biomass using cultivation media and harvesting biomass using a plurality of mechanical and chemical dewatering and extraction techniques and the like, by any of the systems, devices and/or methods described herein.

These and other changes can be made in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method of manufacturing discrete biomass receptacles with at least one of smooth and faceted sides that are usable to perform processes including cultivating algae for biofuel production and/or processing algae for biofuel production, the method comprising:
creating an algae based material from cultivating algae for biofuel production and/or processing algae for biofuel production;
heating the algae based material; and
extruding the algae base material using a three-dimensional printer, the extruding including extruding the algae based material in a concentric pattern in the z-axis to form the biomass receptacles, such that the heating of the algae based material enhances the extrusion.

2. The method according claim 1, wherein the extruding step includes forming a bottom of the biomass receptacles separately from a remainder of the biomass receptacles.

3. The method according claim 2, further comprising adding the bottom to the remainder of each biomass receptacles to form complete biomass receptacles usable for cultivating algae for biofuel production and/or processing algae for biofuel production.

4. The method according to claim 1, wherein the extruding includes forming at least one of an exterior biomass receptacle component and an interior lining for the biomass receptacle.

5. The apparatus according to claim 1, wherein the extruding includes forming at least one of an exterior biomass receptacle component and an interior lining for the biomass receptacle from non-algae material.

6. The apparatus according to claim 4, further comprising, during production or postproduction, forming the exterior receptacle component to be tinted red or blue in order to promote photoautotrophic algae growth within the biomass receptacles.

7. The apparatus according to claim 4, further comprising embedding within the exterior biomass receptacle component at least one of tubes, sensors and associated wires, harnesses, and guide paths.

8. The apparatus according to claim 4, further comprising embedding within the internal receptacle liner at least one of tubes, sensors and associated wires, harnesses, and guide paths.
